(19) United States
(12) Patent Application Publication
(10) Pub. No.: US 2011/0312049 A1
(43) Pub. Date: Dec. 22, 2011

(54) MICROORGANISMS AND METHODS FOR THE PRODUCTION OF ETHYLENE GLYCOL

(76) Inventors: Robin E. Osterhout, San Diego, CA (US); Priti Pharkya, San Diego, CA (US); Anthony P. Burgard, Bellefonte, PA (US)

(21) Appl. No.: 13/086,295

(22) Filed: Apr. 13, 2011

Related U.S. Application Data
(60) Provisional application No. 61/323,650, filed on Apr. 13, 2010.

(51) Int. Cl. C12P 7/18; C12N 1/00; C12N 1/15; C12N 1/21; C12N 1/19

(52) U.S. Cl. 435/158; 435/252.33; 435/252.31; 435/252.32; 435/252.35; 435/252.34; 435/252.3; 435/254.21; 435/254.23; 435/254.3; 435/254.9; 435/254.2; 435/254.11; 435/243

(57) ABSTRACT

The invention provides non-naturally occurring microbial organisms having an ethylene glycol pathway. The invention additionally provides methods of using such organisms to produce ethylene glycol.

This application claims the benefit of priority of U.S. Provisional application Ser. No. 61/323,650, filed Apr. 13, 2010, the entire contents of which are incorporated herein by reference.

### BACKGROUND OF THE INVENTION

The present invention relates generally to biosynthetic processes, and more specifically to organisms having ethylene glycol biosynthetic capability.

Ethylene glycol is a chemical commonly used in many commercial and industrial applications including production of antifreezes and coolants. Ethylene glycol is also used as a raw material in the production of a wide range of products including polyester fibers for clothes, upholstery, carpet and pillows; fiberglass used in products such as jet skis, bathtubs, and bowling balls; and polyethylene terephthalate resin used in packaging film and bottles. Around 82% of ethylene glycol consumed worldwide is used in the production of polyester fibres, resins and films. Strong growth in polyester demand has led to global growth rates of 5-6%/year for ethylene glycol. The second largest market for ethylene is antifreeze formulations.

Typically, in the manufacture of ethylene glycol, ethylene oxide is first produced by the oxidation of ethylene in the presence of oxygen or air and a silver oxide catalyst. A crude ethylene glycol mixture is then produced by the hydrolysis of ethylene oxide with water under pressure. Fractional distillation under vacuum is used to separate the ethylene glycol from the higher glycols. Ethylene glycol was previously manufactured by the hydrolysis of ethylene oxide, which was produced via ethylene chlorohydrin but this method has been superseded by the direct oxidation route. Ethylene glycol is a colorless, odorless, viscous, hygroscopic sweet-tasting liquid and is classified as harmful by the EC Dangerous Substances Directive.

Microbial organisms and methods for effectively producing commercial quantities of ethylene glycol are described herein and include related advantages.

### SUMMARY OF INVENTION

The invention provides non-naturally occurring microbial organisms containing ethylene glycol pathways comprising at least one exogenous nucleic acid encoding an ethylene glycol pathway enzyme expressed in a sufficient amount to produce ethylene glycol. The invention additionally provides methods of using such microbial organisms to produce ethylene glycol, by culturing a non-naturally occurring microbial organism containing ethylene glycol pathways as described herein under conditions and for a sufficient period of time to produce ethylene glycol.

### BRIEF DESCRIPTION OF THE DRAWINGS

FIG. 1 shows exemplary pathways for production of ethylene glycol. Enzymes for transformation of identified substrates to products include: 1) Serine aminotransferase, 2) Serine oxidoreductase (deaminating), 3) Hydroxypyruvate decarboxylase, 4) Glycolaldehyde reductase, 5) Serine decarboxylase, 6) Ethanolamine aminotransferase, 7) Ethanolamine oxidoreductase (deaminating), 8) Hydroxypyruvate reductase, 9) Glycerate decarboxylase, 10) 3-Phosphoglycerate phosphatase, 11) Glycerate kinase, 12) 2-Phosphoglycerate phosphatase, 13) Glycerate-2-kinase and 14) Glyceraldehyde dehydrogenase.

FIG. 2 shows an exemplary pathway for production of ethylene glycol. Enzymes for transformation of identified substrates to products include: 1) Glyoxylate carboligase, 2) Hydroxypyruvate isomerase, 3) Hydroxypyruvate decarboxylase, 4) Glycolaldehyde reductase and 5) Glycerate dehydrogenase.

FIG. 3 shows exemplary pathways for production of ethylene glycol. Enzymes for transformation of identified substrates to products include: 1) Glyoxylate reductase, 2) Glycolyl-CoA transferase, 3) Glycolyl-CoA synthetase, 4) Glycolyl-CoA reductase (aldehyde forming), 5) Glycolaldehyde reductase, 6) Glycolate reductase, 7) Glycolate kinase, 8) Phosphotransglycolylase, 9) Glycolylphosphate reductase and 10) Glycolyl-CoA reductase (alcohol forming).

### DETAILED DESCRIPTION OF THE INVENTION

The present invention is directed to the design and production of cells and organisms having biosynthetic production capabilities for ethylene glycol. The invention, in particular, relates to the design of microbial organism capable of producing ethylene glycol by introducing one or more nucleic acids encoding an ethylene glycol pathway enzyme.

In one embodiment, the invention utilizes in silico stoichiometric models of Escherichia coli metabolism that identify metabolic designs for biosynthetic production of ethylene glycol. The results described herein indicate that metabolic pathways can be designed and recombinantly engineered to achieve the biosynthesis of ethylene glycol in Escherichia coli and other cells or organisms. Biosynthetic production of ethylene glycol, for example, for the in silico designs can be confirmed by construction of strains having the designed metabolic genotype. These metabolically engineered cells or organisms also can be subjected to adaptive evolution to further augment ethylene glycol biosynthesis, including under conditions approaching theoretical maximum growth.

In certain embodiments, the ethylene glycol biosynthesis characteristics of the designed strains make them genetically stable and particularly useful in continuous bioprocesses. Separate strain design strategies were identified with incorporation of different non-native or heterologous reaction capabilities into E. coli or other host organisms leading to ethylene glycol producing metabolic pathways from either serine, 3-phosphoglycerate or glyoxylate. In silico metabolic designs were identified that resulted in the biosynthesis of ethylene glycol in microorganisms from each of these substrates or metabolic intermediates.

Strains identified via the computational component of the platform can be put into actual production by genetically engineering any of the predicted metabolic alterations, which lead to the biosynthetic production of ethylene glycol or other intermediate and/or downstream products. In yet a further embodiment, strains exhibiting biosynthetic production of these compounds can be further subjected to adaptive evolution to further augment product biosynthesis. The levels of product biosynthesis yield following adaptive evolution also can be predicted by the computational component of the system.

The maximum theoretical ethylene glycol yield from glucose is 2.4 mol/mol (0.834 g/g), according to the equation:

C₆H₁₂O₆+1.2H₂O→2.4C₂H₆O₂+1.2CO₂

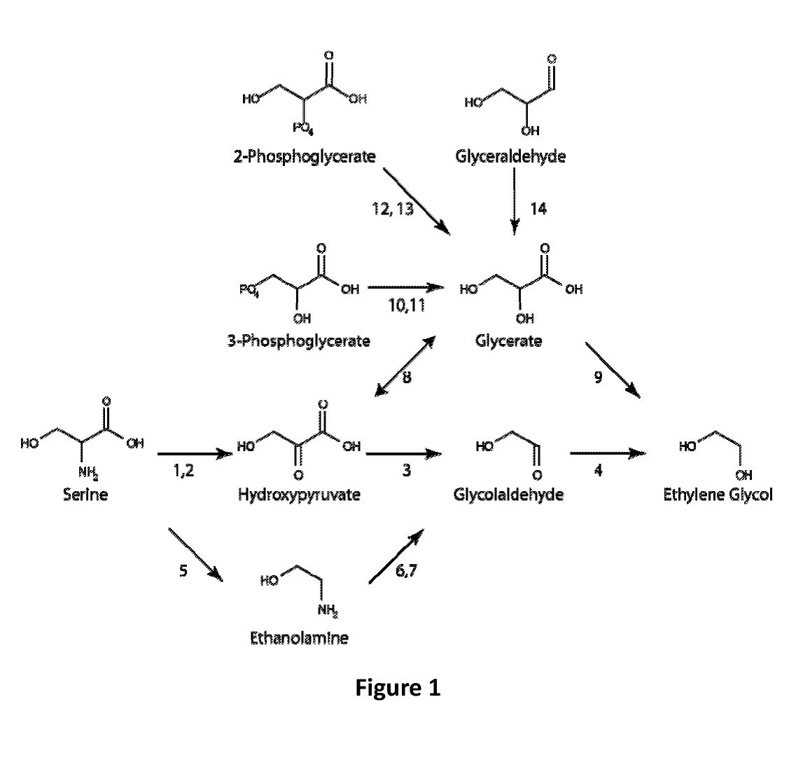

The pathways presented in FIGS. 1-3 achieve a yield of 2 moles ethylene glycol per mole of glucose utilized. Increasing product yields to 2.4 mol/mol is possible if cells are capable of fixing CO₂ through pathways such as the reductive TCA cycle or the Wood-Ljungdahl pathway.

As used herein, the term “non-naturally occurring” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within an ethylene glycol biosynthetic pathway.

A metabolic modification refers to a biochemical reaction that is altered from its naturally occurring state. Therefore, non-naturally occurring microorganisms can have genetic modifications to nucleic acids encoding metabolic polypeptides, or functional fragments thereof. Exemplary metabolic modifications are disclosed herein.

As used herein, the term “ethylene glycol,” having the molecular formula C₂H₆O₂ and a molecular mass of 62.068 g/mol (see FIGS. 1-3) (IUPAC name ethane-1,2-diol) is used interchangeably throughout with monoethylene glycol, MEG, and 1,2-ethanediol. In its pure form, ethylene glycol is an odorless, colorless, syrupy, sweet-tasting liquid. Ethylene glycol is widely used as an antifreeze in automobiles, as a medium for convective heat transfer in cooling systems and as a precursor to polyester fibers and resins. For example, polyethylene terephthalate, which is used to make plastic bottles, is prepared from ethylene glycol. Other known uses for ethylene glycol include use as a desiccant, as a chemical intermediate in the manufacture of capacitors, as an additive to prevent corrosion and as a protecting group for carbonyl groups in organic synthesis.

As used herein, the term “isolated” when used in reference to a microbial organism is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsisted in non-naturally occurring environments. Specific examples of isolated microbial organisms include partially pure microbes, substantially pure microbes and microbes cultured in a medium that is non-naturally occurring.

As used herein, the terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “CoA” or “coenzyme A” is intended to mean an organic cofactor or prosthetic group (nonprotein portion of an enzyme) whose presence is required for the activity of many enzymes (the apoenzyme) to form an active enzyme system. Coenzyme A functions in certain condensing enzymes, acts in acetyl or other acyl group transfer and in fatty acid synthesis and oxidation, pyruvate oxidation and in other acetylation.

As used herein, the term “substantially anaerobic” when used in reference to a culture or growth condition is intended to mean that the amount of oxygen is less than about 10% of saturation for dissolved oxygen in liquid media. The term also is intended to include sealed chambers of liquid or solid medium maintained with an atmosphere of less than about 1% oxygen.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid of the invention can utilize either or both a heterologous or homologous encoding nucleic acid.

It is understood that when more than one exogenous nucleic acid is included in a microbial organism that the more than one exogenous nucleic acids refers to the referenced encoding nucleic acid or biosynthetic activity, as discussed above. It is further understood, as disclosed herein, that such more than one exogenous nucleic acids can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

The non-naturally occurring microbial organisms of the invention can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms of the invention having ethylene glycol biosynthetic capability, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off Those skilled in the art will know what modifications can be made to the above parameters to either increase or decrease the stringency of the comparison, for example, and determine the relatedness of two or more sequences.

In some embodiments, the invention provides a non-naturally occurring microbial organism, including a microbial organism having an ethylene glycol pathway having at least one exogenous nucleic acid encoding an ethylene glycol pathway enzyme expressed in a sufficient amount to produce ethylene glycol, the ethylene glycol pathway including a serine aminotransferase, a serine oxidoreductase (deaminating), a hydroxypyruvate decarboxylase, a glycolaldehyde reductase, a serine decarboxylase, an ethanolamine aminotransferase, an ethanolamine oxidoreductase (deaminating), a hydroxypyruvate reductase or a glycerate decarboxylase (see steps 1-9 of FIG. 1). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a serine aminotransferase or a serine oxidoreductase (deaminating); a hydroxypyruvate decarboxylase, and a glycolaldehyde reductase (see steps 1/2, 3 and 4 of FIG. 1). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a serine aminotransferase or a serine oxidoreductase (deaminating); a hydroxypyruvate reductase, and a glycerate decarboxylase (see steps 1/2, 8, and 9 of FIG. 1). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a serine decarboxylase; an ethanolamine aminotransferase or an ethanolamine oxidoreductase (deaminating), and a glycolaldehyde reductase (see steps 5, 6/7 and 4 of FIG. 1).

In some embodiments, the invention provides a non-naturally occurring microbial organism, including a microbial organism having an ethylene glycol pathway having at least one exogenous nucleic acid encoding an ethylene glycol pathway enzyme expressed in a sufficient amount to produce ethylene glycol, the ethylene glycol pathway including a hydroxypyruvate decarboxylase, glycolaldehyde reductase, a hydroxypyruvate reductase, a glycerate decarboxylase, a 3-phosphoglycerate phosphatase, a glycerate kinase, a 2-phosphoglycerate phosphatase, a glycerate-2-kinase or a glyceraldehyde dehydrogenase (see steps 3, 4, and 8-14 of FIG. 1). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a hydroxypyruvate reductase; a hydroxypyruvate decarboxylase, and a glycolaldehyde reductase (see steps 8, 3 and 4 of FIG. 1). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a 3-phosphoglycerate phosphatase or a glycerate kinase; a hydroxypyruvate reductase; a hydroxypyruvate decarboxylase, and a glycolaldehyde reductase (see steps 10/11, 8, 3 and 4 of FIG. 1). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a 2-phosphoglycerate phosphatase or a glycerate-2-kinase; a hydroxypyruvate reductase; a hydroxypyruvate decarboxylase, and a glycolaldehyde reductase (see steps 12/13, 8, 3 and 4 of FIG. 1). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a glyceraldehyde dehydrogenase, a hydroxypyruvate reductase, a hydroxypyruvate decarboxylase and a glycolaldehyde reductase (see steps 14, 8, 3 and 4 of FIG. 1).

In some embodiments, the invention provides a non-naturally occurring microbial organism, including a microbial organism having an ethylene glycol pathway having at least one exogenous nucleic acid encoding an ethylene glycol pathway enzyme expressed in a sufficient amount to produce ethylene glycol, the ethylene glycol pathway including a glycerate decarboxylase (see step 9 of FIG. 1). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a 3-phosphoglycerate phosphatase or a glycerate kinase and a glycerate decarboxylase (see steps 10/11 and 9 of FIG. 1). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a 2-phosphoglycerate phosphatase, a glycerate-2-kinase and a glycerate decarboxylase (see steps 12/13, 9 of FIG. 1). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a, a glyceraldehyde dehydrogenase and a glycerate decarboxylase (see steps 14 and 9 of FIG. 1).

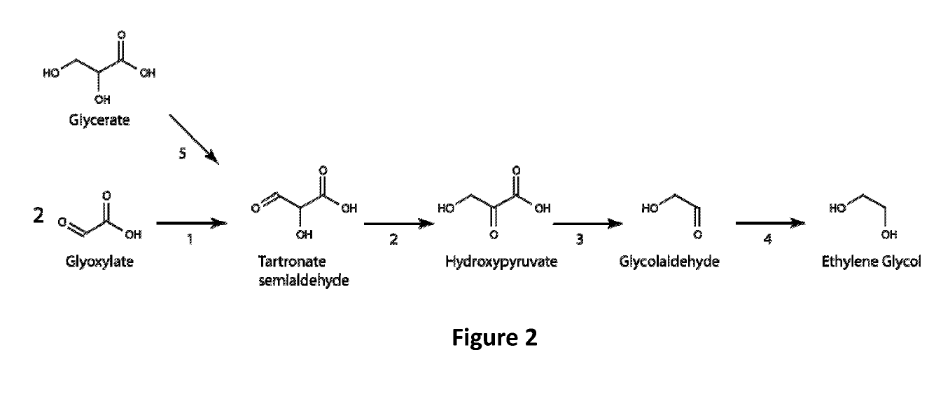

In some embodiments, the invention provides a non-naturally occurring microbial organism, including a microbial organism having an ethylene glycol pathway having at least one exogenous nucleic acid encoding an ethylene glycol pathway enzyme expressed in a sufficient amount to produce ethylene glycol, the ethylene glycol pathway including a glyoxylate carboligase, a hydroxypyruvate isomerase, a hydroxypyruvate decarboxylase, a glycolaldehyde reductase or a glycerate dehydrogenase (see steps 1, 2, 3, 4 or 5 of FIG. 2). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a glyoxylate carboligase, a hydroxypyruvate isomerase, a hydroxypyruvate decarboxylase and a glycolaldehyde reductase (see steps 1, 2, 3 and 4 of FIG. 2). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a glycerate dehydrogenase, a hydroxypyruvate isomerase, a hydroxypyruvate decarboxylase and a glycolaldehyde reductase (see steps 5, 2, 3 and 4 of FIG. 2). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a 3-phosphoglycerate phosphatase or a glycerate kinase; a glycerate dehydrogenase; a hydroxypyruvate isomerase; a hydroxypyruvate decarboxylase; and a glycolaldehyde reductase (see steps 10/11 of FIG. 1 and steps 5, 2, 3 and 4 of FIG. 2). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a 2-phosphoglycerate phosphatase or a glycerate-2-kinase; a glycerate dehydrogenase; a hydroxypyruvate isomerase; a hydroxypyruvate decarboxylase; and a glycolaldehyde reductase (see steps 12/13 of FIG. 1 and steps 5, 2, 3 and 4 of FIG. 2). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a glyceraldehyde dehydrogenase, a glycerate dehydrogenase, a hydroxypyruvate isomerase, a hydroxypyruvate decarboxylase and a glycolaldehyde reductase (see step 14 of FIG. 1 and steps 5, 2, 3 and 4 of FIG. 2).

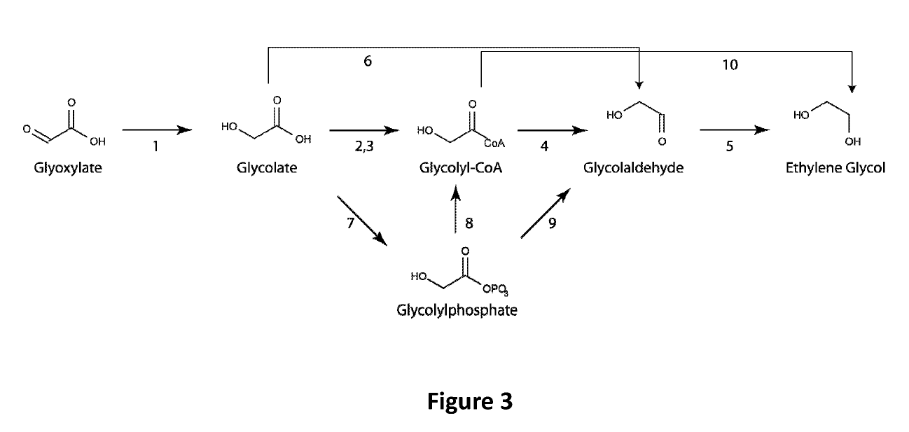

In some embodiments, the invention provides a non-naturally occurring microbial organism, including a microbial organism having an ethylene glycol pathway having at least one exogenous nucleic acid encoding an ethylene glycol pathway enzyme expressed in a sufficient amount to produce ethylene glycol, the ethylene glycol pathway including a glyoxylate reductase, a glycolyl-CoA transferase, a glycolyl-CoA synthetase, a glycolyl-CoA reductase (aldehyde forming), a glycolaldehyde reductase, a glycolate reductase, a glycolate kinase, a phosphotransglycolylase, a glycolylphosphate reductase or a glycolyl-CoA reductase (alcohol forming) (see steps 1-10 of FIG. 3). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a glyoxylate reductase; a glycolyl-CoA transferase or a glycolyl-CoA synthetase; a glycolyl-CoA reductase (aldehyde forming), and a glycolaldehyde reductase (see steps 1, 2/3, 4 and 5 of FIG. 3). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a glyoxylate reductase; a glycolate reductase, and a glycolaldehyde reductase (see steps 1, 6 and 5 of FIG. 3). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a glyoxylate reductase; a glycolyl-CoA transferase or a glycolyl-CoA synthetase, and a glycolyl-CoA reductase (alcohol forming) (see steps 1, 2/3 and 10 of FIG. 3). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a glyoxylate reductase, a glycolate kinase, a phosphotransglycolylase, glycolyl-CoA reductase (aldehyde forming) and a glycolaldehyde reductase (see steps 1, 7, 8, 4 and 5 of FIG. 3). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a glyoxylate reductase, a glycolate kinase, a phosphotransglycolylase and a glycolyl-CoA reductase (alcohol forming) (see steps 1, 7, 8 and 10 of FIG. 3). In one aspect, the non-naturally occurring microbial organism includes a microbial organism having an ethylene glycol pathway having at least one exogenous nucleic acid encoding ethylene glycol pathway enzymes expressed in a sufficient amount to produce ethylene glycol, the ethylene glycol pathway including a glyoxylate reductase, glycolate kinase, a glycolylphosphate reductase and a glycolaldehyde reductase (see steps 1, 7, 9 and 5 of FIG. 3).

In an additional embodiment, the invention provides a non-naturally occurring microbial organism having an ethylene glycol pathway, wherein the non-naturally occurring microbial organism comprises at least one exogenous nucleic acid encoding an enzyme or protein that converts a substrate to a product selected from the group consisting of serine to hydroxypyruvate, hydroxypyruvate to glycolaldehyde, glycolaldehyde to ethylene glycol, serine to ethanolamine, ethanolamine to glycolaldehyde, 3-phosphoglycerate to glycerate; 2-phosphoglycerate to glycerate, glyceraldehyde to glycerate, glycerate to hydroxypyruvate, hydroxypyruvate to glycerate, glycerate to ethylene glycol, glycerate to tartonate semialdehyde, glyoxylate to tartronate semialdehyde, tartronate semialdehyde to hydroxypyruvate, glyoxylate to glycolate, glycolate to glycolaldehyde, glycolate to glycolylphosphate, glycolate to glycolyl-CoA, glycolyl-CoA to ethylene glycol, glycolyl-CoA to glycolaldehyde, glycolylphosphate to glycolyl-CoA and glycolylphosphate to glycolaldehyde. One skilled in the art will understand that these are merely exemplary and that any of the substrate-product pairs disclosed herein suitable to produce a desired product and for which an appropriate activity is available for the conversion of the substrate to the product can be readily determined by one skilled in the art based on the teachings herein. Thus, the invention provides a non-naturally occurring microbial organism containing at least one exogenous nucleic acid encoding an enzyme or protein, where the enzyme or protein converts the substrates and products of an ethylene glycol pathway, such as that shown in FIGS. 1-3.

While generally described herein as a microbial organism that contains an ethylene glycol pathway, it is understood that the invention additionally provides a non-naturally occurring microbial organism comprising at least one exogenous nucleic acid encoding an ethylene glycol pathway enzyme expressed in a sufficient amount to produce an intermediate of an ethylene glycol pathway. For example, as disclosed herein, an ethylene glycol pathway is exemplified in FIGS. 1-3. Therefore, in addition to a microbial organism containing an ethylene glycol pathway that produces ethylene glycol, the invention additionally provides a non-naturally occurring microbial organism comprising at least one exogenous nucleic acid encoding an ethylene glycol pathway enzyme, where the microbial organism produces an ethylene glycol pathway intermediate, for example, hydroxypyruvate, ethanolamine, glycolaldehyde, glycerate, tartronate semialdehyde, glycolate, glycolylphosphate or glycolyl-CoA.

It is understood that any of the pathways disclosed herein, as described in the Examples and exemplified in the Figures, including the pathways of FIGS. 1-3, can be utilized to generate a non-naturally occurring microbial organism that produces any pathway intermediate or product, as desired. As disclosed herein, such a microbial organism that produces an intermediate can be used in combination with another microbial organism expressing downstream pathway enzymes to produce a desired product. However, it is understood that a non-naturally occurring microbial organism that produces an ethylene glycol pathway intermediate can be utilized to produce the intermediate as a desired product.

The invention is described herein with general reference to the metabolic reaction, reactant or product thereof, or with specific reference to one or more nucleic acids or genes encoding an enzyme associated with or catalyzing, or a protein associated with, the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction, and reference to any of these metabolic constituents also references the gene or genes encoding the enzymes that catalyze or proteins involved in the referenced reaction, reactant or product. Likewise, given the well known fields of metabolic biochemistry, enzymology and genomics, reference herein to a gene or encoding nucleic acid also constitutes a reference to the corresponding encoded enzyme and the reaction it catalyzes or a protein associated with the reaction as well as the reactants and products of the reaction.

As disclosed herein, the intermediates glycerate, tartonate semialdehyde, hydroxypyruvate and glyoxylate, as well as other intermediates, are carboxylic acids, which can occur in various ionized forms, including fully protonated, partially protonated, and fully deprotonated forms. Accordingly, the suffix “-ate,” or the acid form, can be used interchangeably to describe both the free acid form as well as any deprotonated form, in particular since the ionized form is known to depend on the pH in which the compound is found. It is understood that carboxylate products or intermediates includes ester forms of carboxylate products or pathway intermediates, such as O-carboxylate and S-carboxylate esters. O- and S-carboxylates can include lower alkyl, that is C1 to C6, branched or straight chain carboxylates. Some such O- or S-carboxylates include, without limitation, methyl, ethyl, n-propyl, n-butyl, i-propyl, sec-butyl, and tert-butyl, pentyl, hexyl O- or S-carboxylates, any of which can further possess an unsaturation, providing for example, propenyl, butenyl, pentyl, and hexenyl O- or S-carboxylates. O-carboxylates can be the product of a biosynthetic pathway. Exemplary O-carboxylates accessed via biosynthetic pathways can include, without limitation, methyl glycerate, ethyl glycerate, n-propyl glycerate, methyl tartonate semialdehyde, ethyl tartonate semialdehyde, n-propyl tartonate semialdehyde, methyl hydroxypyruvate, ethyl hydroxypyruvate, n-propyl hydroxypyruvate, methyl glyoxylate, ethyl glyoxylate, and n-propyl glyoxylate. Other biosynthetically accessible O-carboxylates can include medium to long chain groups, that is C7-C22, O-carboxylate esters derived from fatty alcohols, such heptyl, octyl, nonyl, decyl, undecyl, lauryl, tridecyl, myristyl, pentadecyl, cetyl, palmitolyl, heptadecyl, stearyl, nonadecyl, arachidyl, heneicosyl, and behenyl alcohols, any one of which can be optionally branched and/or contain unsaturations. O-carboxylate esters can also be accessed via a biochemical or chemical process, such as esterification of a free carboxylic acid product or transesterification of an O- or S-carboxylate. S-carboxylates are exemplified by CoA S-esters, cysteinyl S-esters, alkylthioesters, and various aryl and heteroaryl thioesters.

The non-naturally occurring microbial organisms of the invention can be produced by introducing expressible nucleic acids encoding one or more of the enzymes or proteins participating in one or more ethylene glycol biosynthetic pathways. Depending on the host microbial organism chosen for biosynthesis, nucleic acids for some or all of a particular ethylene glycol biosynthetic pathway can be expressed. For example, if a chosen host is deficient in one or more enzymes or proteins for a desired biosynthetic pathway, then expressible nucleic acids for the deficient enzyme(s) or protein(s) are introduced into the host for subsequent exogenous expression. Alternatively, if the chosen host exhibits endogenous expression of some pathway genes, but is deficient in others, then an encoding nucleic acid is needed for the deficient enzyme(s) or protein(s) to achieve ethylene glycol biosynthesis. Thus, a non-naturally occurring microbial organism of the invention can be produced by introducing exogenous enzyme or protein activities to obtain a desired biosynthetic pathway or a desired biosynthetic pathway can be obtained by introducing one or more exogenous enzyme or protein activities that, together with one or more endogenous enzymes or proteins, produces a desired product such as ethylene glycol.

Host microbial organisms can be selected from, and the non-naturally occurring microbial organisms generated in, for example, bacteria, yeast, fungus or any of a variety of other microorganisms applicable to fermentation processes. Exemplary bacteria include species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizobus oryzae, Yarrowia lipolytica, and the like. E. coli is a particularly useful host organism since it is a well characterized microbial organism suitable for genetic engineering. Other particularly useful host organisms include yeast such as Saccharomyces cerevisiae. It is understood that any suitable microbial host organism can be used to introduce metabolic and/or genetic modifications to produce a desired product.

Depending on the ethylene glycol biosynthetic pathway constituents of a selected host microbial organism, the non-naturally occurring microbial organisms of the invention will include at least one exogenously expressed ethylene glycol pathway-encoding nucleic acid and up to all encoding nucleic acids for one or more ethylene glycol biosynthetic pathways. For example, ethylene glycol biosynthesis can be established in a host deficient in a pathway enzyme or protein through exogenous expression of the corresponding encoding nucleic acid. In a host deficient in all enzymes or proteins of an ethylene glycol pathway, exogenous expression of all enzyme or proteins in the pathway can be included, although it is understood that all enzymes or proteins of a pathway can be expressed even if the host contains at least one of the pathway enzymes or proteins. For example, exogenous expression of all enzymes or proteins in a pathway for production of ethylene glycol can be included, such as, a serine aminotransferase, a serine oxidoreductase (deaminating), a hydroxypyruvate decarboxylase, and a glycolaldehyde reductase.

Given the teachings and guidance provided herein, those skilled in the art will understand that the number of encoding nucleic acids to introduce in an expressible form will, at least, parallel the ethylene glycol pathway deficiencies of the selected host microbial organism. Therefore, a non-naturally occurring microbial organism of the invention can have one, two, three, four, five, six, seven, eight, nine or ten up to all nucleic acids encoding the enzymes or proteins constituting an ethylene glycol biosynthetic pathway disclosed herein. In some embodiments, the non-naturally occurring microbial organisms also can include other genetic modifications that facilitate or optimize ethylene glycol biosynthesis or that confer other useful functions onto the host microbial organism. One such other functionality can include, for example, augmentation of the synthesis of one or more of the ethylene glycol pathway precursors such as glycolaldehyde, hydroxypyruvate, ethanolamine, glycerate, tartonate semialdehyde, glycolate, glycolyl-CoA or glycolylphosphate.

Generally, a host microbial organism is selected such that it produces the precursor of an ethylene glycol pathway, either as a naturally produced molecule or as an engineered product that either provides de novo production of a desired precursor or increased production of a precursor naturally produced by the host microbial organism. For example, serine is produced naturally in a host organism such as E. coli. A host organism can be engineered to increase production of a precursor, as disclosed herein. In addition, a microbial organism that has been engineered to produce a desired precursor can be used as a host organism and further engineered to express enzymes or proteins of an ethylene glycol pathway.

In some embodiments, a non-naturally occurring microbial organism of the invention is generated from a host that contains the enzymatic capability to synthesize ethylene glycol. In this specific embodiment it can be useful to increase the synthesis or accumulation of an ethylene glycol pathway product to, for example, drive ethylene glycol pathway reactions toward ethylene glycol production. Increased synthesis or accumulation can be accomplished by, for example, overexpression of nucleic acids encoding one or more of the above-described ethylene glycol pathway enzymes or proteins. Over expression the enzyme or enzymes and/or protein or proteins of the ethylene glycol pathway can occur, for example, through exogenous expression of the endogenous gene or genes, or through exogenous expression of the heterologous gene or genes. Therefore, naturally occurring organisms can be readily generated to be non-naturally occurring microbial organisms of the invention, for example, producing ethylene glycol, through overexpression of one, two, three, four five, six, seven, eight, nine or 10, that is, up to all nucleic acids encoding ethylene glycol biosynthetic pathway enzymes or proteins. In addition, a non-naturally occurring organism can be generated by mutagenesis of an endogenous gene that results in an increase in activity of an enzyme in the ethylene glycol biosynthetic pathway.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

It is understood that, in methods of the invention, any of the one or more exogenous nucleic acids can be introduced into a microbial organism to produce a non-naturally occurring microbial organism of the invention. The nucleic acids can be introduced so as to confer, for example, an ethylene glycol biosynthetic pathway onto the microbial organism. Alternatively, encoding nucleic acids can be introduced to produce an intermediate microbial organism having the biosynthetic capability to catalyze some of the required reactions to confer ethylene glycol biosynthetic capability. For example, a non-naturally occurring microbial organism having an ethylene glycol biosynthetic pathway can comprise at least two exogenous nucleic acids encoding desired enzymes or proteins, such as the combination of a hydroxypyruvate decarboxylase and a glycolaldehyde reductase, or alternatively a serine decarboxylase and an ethanolamine oxidoreductase (deaminating), or alternatively a glyoxylate carboligase and a hydroxypyruvate isomerase, or alternatively a glycolyl-CoA reductase (aldehyde forming) and a glycolaldehyde reductase, or alternatively 2-phosphoglycerate phosphatase and glycoaldehyde reductase and the like. Thus, it is understood that any combination of two or more enzymes or proteins of a biosynthetic pathway can be included in a non-naturally occurring microbial organism of the invention. Similarly, it is understood that any combination of three or more enzymes or proteins of a biosynthetic pathway can be included in a non-naturally occurring microbial organism of the invention, for example, a serine oxidoreductase (deaminating), a hydroxypyruvate decarboxylase, and a glycolaldehyde reductase, or alternatively, a glycerate kinase; a hydroxypyruvate reductase and a hydroxypyruvate decarboxylase, or alternatively a 3-phosphoglycerate phosphatase, a glycerate kinase and a glycerate decarboxylase, or alternatively a glyoxylate carboligase, a hydroxypyruvate isomerase and a hydroxypyruvate decarboxylase, or alternatively a glycolyl-CoA transferase, a glycolyl-CoA reductase (aldehyde forming) and a glycolaldehyde reductase, or alternatively a glyoxylate reductase, a glycolyl-CoA transferase and a glycolyl-CoA reductase (alcohol forming), and so forth, as desired, so long as the combination of enzymes and/or proteins of the desired biosynthetic pathway results in production of the corresponding desired product. Similarly, any combination of four, a serine decarboxylase, an ethanolamine aminotransferase, an ethanolamine oxidoreductase (deaminating) and a glycolaldehyde reductase, or alternatively a 3-phosphoglycerate phosphatase, a hydroxypyruvate reductase, a hydroxypyruvate decarboxylase and a glycolaldehyde reductase, or alternatively a glyoxylate carboligase, a hydroxypyruvate isomerase, a hydroxypyruvate decarboxylase and a glycolaldehyde reductase, or alternatively a glyoxylate reductase, glycolate kinase, a glycolylphosphate reductase and a glycolaldehyde reductase, or alternatively a glyceraldehyde dehydrogenase, a glycerate dehydrogenase, and a hydroxypyruvate decarboxylase or more enzymes or proteins of a biosynthetic pathway as disclosed herein can be included in a non-naturally occurring microbial organism of the invention, as desired, so long as the combination of enzymes and/or proteins of the desired biosynthetic pathway results in production of the corresponding desired product.

In addition to the biosynthesis of ethylene glycol as described herein, the non-naturally occurring microbial organisms and methods of the invention also can be utilized in various combinations with each other and with other microbial organisms and methods well known in the art to achieve product biosynthesis by other routes. For example, one alternative to produce ethylene glycol other than use of the ethylene glycol producers is through addition of another microbial organism capable of converting an ethylene glycol pathway intermediate to ethylene glycol. One such procedure includes, for example, the fermentation of a microbial organism that produces an ethylene glycol pathway intermediate. The ethylene glycol pathway intermediate can then be used as a substrate for a second microbial organism that converts the ethylene glycol pathway intermediate to ethylene glycol. The ethylene glycol pathway intermediate can be added directly to another culture of the second organism or the original culture of the ethylene glycol pathway intermediate producers can be depleted of these microbial organisms by, for example, cell separation, and then subsequent addition of the second organism to the fermentation broth can be utilized to produce the final product without intermediate purification steps.

In other embodiments, the non-naturally occurring microbial organisms and methods of the invention can be assembled in a wide variety of subpathways to achieve biosynthesis of, for example, ethylene glycol. In these embodiments, biosynthetic pathways for a desired product of the invention can be segregated into different microbial organisms, and the different microbial organisms can be co-cultured to produce the final product. In such a biosynthetic scheme, the product of one microbial organism is the substrate for a second microbial organism until the final product is synthesized. For example, the biosynthesis of ethylene glycol can be accomplished by constructing a microbial organism that contains biosynthetic pathways for conversion of one pathway intermediate to another pathway intermediate or the product. Alternatively, ethylene glycol also can be biosynthetically produced from microbial organisms through co-culture or co-fermentation using two organisms in the same vessel, where the first microbial organism produces an ethylene glycol intermediate and the second microbial organism converts the intermediate to ethylene glycol.

Given the teachings and guidance provided herein, those skilled in the art will understand that a wide variety of combinations and permutations exist for the non-naturally occurring microbial organisms and methods of the invention together with other microbial organisms, with the co-culture of other non-naturally occurring microbial organisms having subpathways and with combinations of other chemical and/or biochemical procedures well known in the art to produce ethylene glycol.

Sources of encoding nucleic acids for an ethylene glycol pathway enzyme or protein can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, Escherichia coli, Rattus norvegicus, Homo sapiens, Drosophila melanogaster, Mus musculus, Sus scrofa, Arabidopsis thaliana, Oryza sativa, Hyphomicrobium methylovorum, Methylobacterium extorquens, Thermotoga maritima, Halobacterium salinarum, Lactococcus lactis, Saccharomyces cerevisiae, Zymomonas mobilis, Acinetobacter sp. Strain M-1, Brassica napus, Beta vulgaris, Geobacillus stearothermophilus, Agrobacterium tumefaciens, Acinetobacter calcoaceticus, Acinetobacter baylyi, Achromobacter denitrificans, Streptococcus thermophilus, Bacillus brevis, Bacillus subtilis, Bacillus megaterium, Enterobacter aerogenes, Ralstonia eutropha, Salmonella enterica, Salmonella typhimurium, Burkholderia ambifaria, Acidaminococcus fermentans, Archaeoglobus fulgidus, Haloarcula marismortui, Pyrobaculum aerophilum str. IM2, Pseudomonas putida, Pseudomonas sp, Rhizobium leguminosarum, Clostridium kluyveri, Clostridium saccharoperbutylacetonicum, Clostridium acetobutylicum, Clostridium beijerinckii, Porphyromonas gingivalis, Leuconostoc mesenteroides, Metallosphaera sedula, Sulfolobus tokodaii, Sulfolobus solfataricus, Sulfolobus acidocaldarius, Nocardia iowensis, Streptomyces griseus, Candida albicans, Schizosaccharomyces pombe, Penicillium chrysogenum, butyrate-producing bacterium L2-50, Haemophilus influenzae, Mycobacterium tuberculosis, Vibrio cholera, Helicobacter pylori, Campylobacter jejuni, Leuconostoc mesenteroides, Chloroflexus aurantiacus, Roseiflexus castenholzii, Erythrobacter sp. NAP1, marine gamma proteobacterium HTCC2080, Simmondsia chinensis, Azospirillum brasilense, Bos Taurus, Clostridium kluyveri DSM 555, Geobacillus thermoglucosidasius, Methanocaldococcus jannaschii, Oryctolagus cuniculus, Oryza sativa, Phaseolus vulgaris, Picrophilus torridus, Pseudomonas aeruginosa, Pyrococcus furiosus, Ralstonia eutropha H16, Staphylococcus aureus, Thermoproteus tenax, Thermus thermophilus, and Zea mays as well as other exemplary species disclosed herein or available as source organisms for corresponding genes. However, with the complete genome sequence available for now more than 550 species (with more than half of these available on public databases such as the NCBI), including 395 microorganism genomes and a variety of yeast, fungi, plant, and mammalian genomes, the identification of genes encoding the requisite ethylene glycol biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations allowing biosynthesis of ethylene glycol described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

In some instances, such as when an alternative ethylene glycol biosynthetic pathway exists in an unrelated species, ethylene glycol biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms may differ. However, given the teachings and guidance provided herein, those skilled in the art also will understand that the teachings and methods of the invention can be applied to all microbial organisms using the cognate metabolic alterations to those exemplified herein to construct a microbial organism in a species of interest that will synthesize ethylene glycol.

Methods for constructing and testing the expression levels of a non-naturally occurring ethylene glycol-producing host can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

Exogenous nucleic acid sequences involved in a pathway for production of ethylene glycol can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to include one or more ethylene glycol biosynthetic pathway encoding nucleic acids as exemplified herein operably linked to expression control sequences functional in the host organism. Expression vectors applicable for use in the microbial host organisms of the invention include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

In some embodiments, the invention provides a method for producing ethylene glycol that includes culturing a non-naturally occurring microbial organism, including a microbial organism having an ethylene glycol pathway, the ethylene glycol pathway including at least one exogenous nucleic acid encoding an ethylene glycol pathway enzyme expressed in a sufficient amount to produce ethylene glycol, the ethylene glycol pathway including a serine aminotransferase, a serine oxidoreductase (deaminating), a hydroxypyruvate decarboxylase, a glycolaldehyde reductase, a serine decarboxylase, an ethanolamine aminotransferase, an ethanolamine oxidoreductase (deaminating), a hydroxypyruvate reductase or a glycerate decarboxylase (see steps 1-9 of FIG. 1). In one aspect, the method includes a microbial organism having an ethylene glycol pathway including a serine aminotransferase or a serine oxidoreductase (deaminating); a hydroxypyruvate decarboxylase, and a glycolaldehyde reductase (see steps 1/2, 3 and 4 of FIG. 1). In one aspect, the method includes a microbial organism having an ethylene glycol pathway including a serine aminotransferase or a serine oxidoreductase (deaminating); a hydroxypyruvate reductase, and a glycerate decarboxylase (see steps 1/2, 8, and 9 of FIG. 1). In one aspect, the method includes a microbial organism having an ethylene glycol pathway including a serine decarboxylase; an ethanolamine aminotransferase or an ethanolamine oxidoreductase (deaminating), and a glycolaldehyde reductase (see steps 5, 6/7 and 4 of FIG. 1).

In some embodiments, the invention provides a method for producing ethylene glycol that includes culturing a non-naturally occurring microbial organism, including a microbial organism having an ethylene glycol pathway, the ethylene glycol pathway including at least one exogenous nucleic acid encoding an ethylene glycol pathway enzyme expressed in a sufficient amount to produce ethylene glycol, the ethylene glycol pathway including a hydroxypyruvate decarboxylase, glycolaldehyde reductase, a hydroxypyruvate reductase, a glycerate decarboxylase, a 3-phosphoglycerate phosphatase, a glycerate kinase, a 2-phosphoglycerate phosphatase, a glycerate-2-kinase or a glyceraldehyde dehydrogenase (see steps 3, 4, and 8-14 of FIG. 1). In one aspect, the method includes a microbial organism having an ethylene glycol pathway including a hydroxypyruvate reductase, a hydroxypyruvate decarboxylase and a glycolaldehyde reductase (see steps 8, 3 and 4 of FIG. 1). In one aspect, the method includes a microbial organism having an ethylene glycol pathway including a 3-phosphoglycerate phosphatase or a glycerate kinase; a hydroxypyruvate reductase; a hydroxypyruvate decarboxylase, and a glycolaldehyde reductase (see steps 10/11, 8, 3 and 4 of FIG. 1). In one aspect, the method includes a microbial organism having an ethylene glycol pathway including a 2-phosphoglycerate phosphatase or a glycerate-2-kinase; a hydroxypyruvate reductase; a hydroxypyruvate decarboxylase, and a glycolaldehyde reductase (see steps 12/13, 8, 3 and 4 of FIG. 1). In one aspect, the method includes a microbial organism having an ethylene glycol pathway including a glyceraldehyde dehydrogenase, a hydroxypyruvate reductase; a hydroxypyruvate decarboxylase, and a glycolaldehyde reductase (see steps 14, 8, 3 and 4 of FIG. 1).

In some embodiments, the invention provides a method for producing ethylene glycol that includes culturing a non-naturally occurring microbial organism, including a microbial organism having an ethylene glycol pathway, the ethylene glycol pathway including at least one exogenous nucleic acid encoding an ethylene glycol pathway enzyme expressed in a sufficient amount to produce ethylene glycol, the ethylene glycol pathway including a glycerate decarboxylase (see step 9 of FIG. 1). In one aspect, the method includes a microbial organism having an ethylene glycol pathway including a 3-phosphoglycerate phosphatase or a glycerate kinase and a glycerate decarboxylase (see steps 10/11 and 9 of FIG. 1). In one aspect, the method includes a microbial organism having an ethylene glycol pathway including a 2-phosphoglycerate phosphatase, a glycerate-2-kinase and a glycerate decarboxylase (see steps 12/13, 9 of FIG. 1). In one aspect, the method includes a microbial organism having an ethylene glycol pathway including a glyceraldehyde dehydrogenase and a glycerate decarboxylase (see steps 14 and 9 of FIG. 1).

In some embodiments, the invention provides a method for producing ethylene glycol that includes culturing a non-naturally occurring microbial organism, including a microbial organism having an ethylene glycol pathway, the ethylene glycol pathway including at least one exogenous nucleic acid encoding an ethylene glycol pathway enzyme expressed in a sufficient amount to produce ethylene glycol, the ethylene glycol pathway including a glyoxylate carboligase, a hydroxypyruvate isomerase, a hydroxypyruvate decarboxylase, a glycolaldehyde reductase or a glycerate dehydrogenase (see steps 1, 2, 3, 4 or 5 of FIG. 2). In one aspect, the method includes a microbial organism having an ethylene glycol pathway including a glyoxylate carboligase, a hydroxypyruvate isomerase, a hydroxypyruvate decarboxylase and a glycolaldehyde reductase (see steps 1, 2, 3 and 4 of FIG. 2). In one aspect, the method includes a microbial organism having an ethylene glycol pathway including a glycerate dehydrogenase, a hydroxypyruvate isomerase, a hydroxypyruvate decarboxylase and a glycolaldehyde reductase (see steps 5, 2, 3 and 4 of FIG. 2). In one aspect, the method includes a microbial organism having an ethylene glycol pathway including a 3-phosphoglycerate phosphatase or a glycerate kinase; a glycerate dehydrogenase; a hydroxypyruvate isomerase; a hydroxypyruvate decarboxylase; and a glycolaldehyde reductase (see steps 10/11 of FIG. 1 and steps 5, 2, 3 and 4 of FIG. 2). In one aspect, the method includes a microbial organism having an ethylene glycol pathway including a 2-phosphoglycerate phosphatase or a glycerate-2-kinase; a glycerate dehydrogenase; a hydroxypyruvate isomerase; a hydroxypyruvate decarboxylase; and a glycolaldehyde reductase (see steps 12/13 of FIG. 1 and steps 5, 2, 3 and 4 of FIG. 2). In one aspect, the method includes a microbial organism having an ethylene glycol pathway including a glyceraldehyde dehydrogenase, a glycerate dehydrogenase, a hydroxypyruvate isomerase, a hydroxypyruvate decarboxylase and a glycolaldehyde reductase (see step 14 of FIG. 1 and steps 5, 2, 3 and 4 of FIG. 2).

In some embodiments, the invention provides a method for producing ethylene glycol that includes culturing a non-naturally occurring microbial organism, including a microbial organism having an ethylene glycol pathway, the ethylene glycol pathway including at least one exogenous nucleic acid encoding an ethylene glycol pathway enzyme expressed in a sufficient amount to produce ethylene glycol, the ethylene glycol pathway including a glyoxylate reductase, a glycolyl-CoA transferase, a glycolyl-CoA synthetase, a glycolyl-CoA reductase (aldehyde forming), a glycolaldehyde reductase, a glycolate reductase, a glycolate kinase, a phosphotransglycolylase, a glycolylphosphate reductase or a glycolyl-CoA reductase (alcohol forming) (see steps 1-10 of FIG. 3). In one aspect, the method includes a microbial organism having an ethylene glycol pathway including a glyoxylate reductase; a glycolyl-CoA transferase or a glycolyl-CoA synthetase; a glycolyl-CoA reductase (aldehyde forming), and a glycolaldehyde reductase (see steps 1, 2/3, 4 and 5 of FIG. 3). In one aspect, the method includes a microbial organism having an ethylene glycol pathway including a glyoxylate reductase; a glycolate reductase, and a glycolaldehyde reductase (see steps 1, 6 and 5 of FIG. 3). In one aspect, the method includes a microbial organism having an ethylene glycol pathway including a glyoxylate reductase; a glycolyl-CoA transferase or a glycolyl-CoA synthetase, and a glycolyl-CoA reductase (alcohol forming) (see steps 1, 2/3 and 10 of FIG. 3). In one aspect, the method includes a microbial organism having an ethylene glycol pathway including a glyoxylate reductase, a glycolate kinase, a phosphotransglycolylase, glycolyl-CoA reductase (aldehyde forming) and a glycolaldehyde reductase (see steps 1, 7, 8, 4 and 5 of FIG. 3). In one aspect, the method includes a microbial organism having an ethylene glycol pathway including a glyoxylate reductase, a glycolate kinase, a phosphotransglycolylase and a glycolyl-CoA reductase (alcohol forming) (see steps 1, 7, 8 and 10 of FIG. 3). In one aspect, the method includes a microbial organism having an ethylene glycol pathway including a glyoxylate reductase, glycolate kinase, a glycolylphosphate reductase and a glycolaldehyde reductase (see steps 1, 7, 9 and 5 of FIG. 3).

Suitable purification and/or assays to test for the production of ethylene glycol can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art. For example, glycolaldehyde reductase activity can be measured by its NADH-dependent glycolaldehyde reduction to ethylene glycol using a molar absorption coefficient of 6.22×10⁻³ M⁻¹ at 340 nm.

The ethylene glycol can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. All of the above methods are well known in the art.

Any of the non-naturally occurring microbial organisms described herein can be cultured to produce and/or secrete the biosynthetic products of the invention. For example, the ethylene glycol producers can be cultured for the biosynthetic production of ethylene glycol.

For the production of ethylene glycol, the recombinant strains are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in United State publication 2009/0047719, filed Aug. 10, 2007. Fermentations can be performed in a batch, fed-batch or continuous manner, as disclosed herein.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

The growth medium can include, for example, any carbohydrate source which can supply a source of carbon to the non-naturally occurring microorganism. Such sources include, for example, sugars such as glucose, xylose, arabinose, galactose, mannose, fructose, sucrose and starch. Other sources of carbohydrate include, for example, renewable feedstocks and biomass. Exemplary types of biomasses that can be used as feedstocks in the methods of the invention include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the microbial organisms of the invention for the production of ethylene glycol.

In addition to renewable feedstocks such as those exemplified above, the ethylene glycol microbial organisms of the invention also can be modified for growth on syngas as its source of carbon. In this specific embodiment, one or more proteins or enzymes are expressed in the ethylene glycol producing organisms to provide a metabolic pathway for utilization of syngas or other gaseous carbon source.

Synthesis gas, also known as syngas or producer gas, is the major product of gasification of coal and of carbonaceous materials such as biomass materials, including agricultural crops and residues. Syngas is a mixture primarily of H₂ and CO and can be obtained from the gasification of any organic feedstock, including but not limited to coal, coal oil, natural gas, biomass, and waste organic matter. Gasification is generally carried out under a high fuel to oxygen ratio. Although largely H₂ and CO, syngas can also include CO₂ and other gases in smaller quantities. Thus, synthesis gas provides a cost effective source of gaseous carbon such as CO and, additionally, CO₂.

The Wood-Ljungdahl pathway catalyzes the conversion of CO and H₂ to acetyl-CoA and other products such as acetate. Organisms capable of utilizing CO and syngas also generally have the capability of utilizing CO₂ and CO₂/H₂ mixtures through the same basic set of enzymes and transformations encompassed by the Wood-Ljungdahl pathway. H₂-dependent conversion of CO₂ to acetate by microorganisms was recognized long before it was revealed that CO also could be used by the same organisms and that the same pathways were involved. Many acetogens have been shown to grow in the presence of CO₂ and produce compounds such as acetate as long as hydrogen is present to supply the necessary reducing equivalents (see for example, Drake, Acetogenesis, pp. 3-60 Chapman and Hall, New York, (1994)). This can be summarized by the following equation:

2CO₂+4H₂+nADP+nPi→CH₃COOH+2H₂O+nATP

Hence, non-naturally occurring microorganisms possessing the Wood-Ljungdahl pathway can utilize CO₂ and H₂ mixtures as well for the production of acetyl-CoA and other desired products.

The Wood-Ljungdahl pathway is well known in the art and consists of 12 reactions which can be separated into two branches: (1) methyl branch and (2) carbonyl branch. The methyl branch converts syngas to methyl-tetrahydrofolate (methyl-THF) whereas the carbonyl branch converts methyl-THF to acetyl-CoA. The reactions in the methyl branch are catalyzed in order by the following enzymes or proteins: ferredoxin oxidoreductase, formate dehydrogenase, formyltetrahydrofolate synthetase, methenyltetrahydrofolate cyclodehydratase, methylenetetrahydrofolate dehydrogenase and methylenetetrahydrofolate reductase. The reactions in the carbonyl branch are catalyzed in order by the following enzymes or proteins: methyltetrahydrofolate:corrinoid protein methyltransferase (for example, AcsE), corrinoid iron-sulfur protein, nickel-protein assembly protein (for example, AcsF), ferredoxin, acetyl-CoA synthase, carbon monoxide dehydrogenase and nickel-protein assembly protein (for example, CooC). Following the teachings and guidance provided herein for introducing a sufficient number of encoding nucleic acids to generate an ethylene glycol pathway, those skilled in the art will understand that the same engineering design also can be performed with respect to introducing at least the nucleic acids encoding the Wood-Ljungdahl enzymes or proteins absent in the host organism. Therefore, introduction of one or more encoding nucleic acids into the microbial organisms of the invention such that the modified organism contains the complete Wood-Ljungdahl pathway will confer syngas utilization ability.

Additionally, the reductive (reverse) tricarboxylic acid cycle coupled with carbon monoxide dehydrogenase and/or hydrogenase activities can also be used for the conversion of CO, CO₂ and/or H₂ to acetyl-CoA and other products such as acetate. Organisms capable of fixing carbon via the reductive TCA pathway can utilize one or more of the following enzymes: ATP citrate-lyase, citrate lyase, aconitase, isocitrate dehydrogenase, alpha-ketoglutarate:ferredoxin oxidoreductase, succinyl-CoA synthetase, succinyl-CoA transferase, fumarate reductase, fumarase, malate dehydrogenase, NAD(P)H:ferredoxin oxidoreductase, carbon monoxide dehydrogenase, and hydrogenase. Specifically, the reducing equivalents extracted from CO and/or H₂ by carbon monoxide dehydrogenase and hydrogenase are utilized to fix CO₂ via the reductive TCA cycle into acetyl-CoA or acetate. Acetate can be converted to acetyl-CoA by enzymes such as acetyl-CoA transferase, acetate kinase/phosphotransacetylase, and acetyl-CoA synthetase. Acetyl-CoA can be converted to several metabolic intermediates including serine, 3-phosphoglycerate, 2-phosphoglycerate, glyceraldehyde and glyoxylate percursors by common central metabolic reactions, and glyceraldehyde-3-phosphate, phosphoenolpyruvate, and pyruvate, by pyruvate:ferredoxin oxidoreductase and the enzymes of gluconeogenesis. Following the teachings and guidance provided herein for introducing a sufficient number of encoding nucleic acids to generate a serine, 3-phosphoglycerate, 2-phosphoglycerate, glyceraldehyde or glyoxylate pathway, those skilled in the art will understand that the same engineering design also can be performed with respect to introducing at least the nucleic acids encoding the reductive TCA pathway enzymes or proteins absent in the host organism. Therefore, introduction of one or more encoding nucleic acids into the microbial organisms of the invention such that the modified organism contains the complete reductive TCA pathway will confer syngas utilization ability.

Accordingly, given the teachings and guidance provided herein, those skilled in the art will understand that a non-naturally occurring microbial organism can be produced that secretes the biosynthesized compounds of the invention when grown on a carbon source such as a carbohydrate. Such compounds include, for example, ethylene glycol and any of the intermediate metabolites in the ethylene glycol pathway. All that is required is to engineer in one or more of the required enzyme or protein activities to achieve biosynthesis of the desired compound or intermediate including, for example, inclusion of some or all of the ethylene glycol biosynthetic pathways. Accordingly, the invention provides a non-naturally occurring microbial organism that produces and/or secretes ethylene glycol when grown on a carbohydrate or other carbon source and produces and/or secretes any of the intermediate metabolites shown in the ethylene glycol pathway when grown on a carbohydrate or other carbon source. The ethylene glycol producing microbial organisms of the invention can initiate synthesis from an intermediate, for example, hydroxypyruvate, ethanolamine, glycolaldehyde, glycerate, tartronate semialdehyde, glycolate, glycolylphosphate or glycolyl-CoA.

The non-naturally occurring microbial organisms of the invention are constructed using methods well known in the art as exemplified herein to exogenously express at least one nucleic acid encoding an ethylene glycol pathway enzyme or protein in sufficient amounts to produce ethylene glycol. It is understood that the microbial organisms of the invention are cultured under conditions sufficient to produce ethylene glycol. Following the teachings and guidance provided herein, the non-naturally occurring microbial organisms of the invention can achieve biosynthesis of ethylene glycol resulting in intracellular concentrations between about 0.1-2000 mM or more. Generally, the intracellular concentration of ethylene glycol is between about 3-1500 mM, particularly between about 5-1250 mM and more particularly between about 8-1000 mM, including about 100 mM, 200 mM, 500 mM, 800 mM, or more. Intracellular concentrations between and above each of these exemplary ranges also can be achieved from the non-naturally occurring microbial organisms of the invention.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. publication 2009/0047719, filed Aug. 10, 2007. Any of these conditions can be employed with the non-naturally occurring microbial organisms as well as other anaerobic conditions well known in the art. Under such anaerobic or substantially anaerobic conditions, the ethylene glycol producers can synthesize ethylene glycol at intracellular concentrations of 5-10 mM or more as well as all other concentrations exemplified herein. It is understood that, even though the above description refers to intracellular concentrations, ethylene glycol producing microbial organisms can produce ethylene glycol intracellularly and/or secrete the product into the culture medium.

In addition to the culturing and fermentation conditions disclosed herein, growth condition for achieving biosynthesis of ethylene glycol can include the addition of an osmoprotectant to the culturing conditions. In certain embodiments, the non-naturally occurring microbial organisms of the invention can be sustained, cultured or fermented as described herein in the presence of an osmoprotectant. Briefly, an osmoprotectant refers to a compound that acts as an osmolyte and helps a microbial organism as described herein survive osmotic stress. Osmoprotectants include, but are not limited to, betaines, amino acids, and the sugar trehalose. Non-limiting examples of such are glycine betaine, praline betaine, dimethylthetin, dimethylslfonioproprionate, 3-dimethylsulfonio-2-methylproprionate, pipecolic acid, dimethylsulfonioacetate, choline, L-carnitine and ectoine. In one aspect, the osmoprotectant is glycine betaine. It is understood to one of ordinary skill in the art that the amount and type of osmoprotectant suitable for protecting a microbial organism described herein from osmotic stress will depend on the microbial organism used. The amount of osmoprotectant in the culturing conditions can be, for example, no more than about 0.1 mM, no more than about 0.5 mM, no more than about 1.0 mM, no more than about 1.5 mM, no more than about 2.0 mM, no more than about 2.5 mM, no more than about 3.0 mM, no more than about 5.0 mM, no more than about 7.0 mM, no more than about 10 mM, no more than about 50 mM, no more than about 100 mM or no more than about 500 mM.

The culture conditions can include, for example, liquid culture procedures as well as fermentation and other large scale culture procedures. As described herein, particularly useful yields of the biosynthetic products of the invention can be obtained under anaerobic or substantially anaerobic culture conditions.

As described herein, one exemplary growth condition for achieving biosynthesis of ethylene glycol includes anaerobic culture or fermentation conditions. In certain embodiments, the non-naturally occurring microbial organisms of the invention can be sustained, cultured or fermented under anaerobic or substantially anaerobic conditions. Briefly, anaerobic conditions refers to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions described herein can be scaled up and grown continuously for manufacturing of ethylene glycol. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of ethylene glycol. Generally, and as with non-continuous culture procedures, the continuous and/or near-continuous production of ethylene glycol will include culturing a non-naturally occurring ethylene glycol producing organism of the invention in sufficient nutrients and medium to sustain and/or nearly sustain growth in an exponential phase. Continuous culture under such conditions can include, for example, growth for 1 day, 2, 3, 4, 5, 6 or 7 days or more. Additionally, continuous culture can include longer time periods of 1 week, 2, 3, 4 or 5 or more weeks and up to several months. Alternatively, organisms of the invention can be cultured for hours, if suitable for a particular application. It is to be understood that the continuous and/or near-continuous culture conditions also can include all time intervals in between these exemplary periods. It is further understood that the time of culturing the microbial organism of the invention is for a sufficient period of time to produce a sufficient amount of product for a desired purpose.

Fermentation procedures are well known in the art. Briefly, fermentation for the biosynthetic production of ethylene glycol can be utilized in, for example, fed-batch fermentation and batch separation; fed-batch fermentation and continuous separation, or continuous fermentation and continuous separation. Examples of batch and continuous fermentation procedures are well known in the art.

In addition to the above fermentation procedures using the ethylene glycol producers of the invention for continuous production of substantial quantities of ethylene glycol, the ethylene glycol producers also can be, for example, simultaneously subjected to chemical synthesis procedures to convert the product to other compounds or the product can be separated from the fermentation culture and sequentially subjected to chemical or enzymatic conversion to convert the product to other compounds, if desired.

To generate better producers, metabolic modeling can be utilized to optimize growth conditions. Modeling can also be used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on cell growth of shifting the metabolism towards more efficient production of ethylene glycol.

One computational method for identifying and designing metabolic alterations favoring biosynthesis of a desired product is the OptKnock computational framework (Burgard et al., Biotechnol. Bioeng. 84:647-657 (2003)). OptKnock is a metabolic modeling and simulation program that suggests gene deletion or disruption strategies that result in genetically stable microorganisms which overproduce the target product. Specifically, the framework examines the complete metabolic and/or biochemical network of a microorganism in order to suggest genetic manipulations that force the desired biochemical to become an obligatory byproduct of cell growth. By coupling biochemical production with cell growth through strategically placed gene deletions or other functional gene disruption, the growth selection pressures imposed on the engineered strains after long periods of time in a bioreactor lead to improvements in performance as a result of the compulsory growth-coupled biochemical production. Lastly, when gene deletions are constructed there is a negligible possibility of the designed strains reverting to their wild-type states because the genes selected by OptKnock are to be completely removed from the genome. Therefore, this computational methodology can be used to either identify alternative pathways that lead to biosynthesis of a desired product or used in connection with the non-naturally occurring microbial organisms for further optimization of biosynthesis of a desired product.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that allow an effective query of the performance limits of metabolic networks and provides methods for solving the resulting mixed-integer linear programming problems. The metabolic modeling and simulation methods referred to herein as OptKnock are described in, for example, U.S. publication 2002/0168654, filed Jan. 10, 2002, in International Patent No. PCT/US02/00660, filed Jan. 10, 2002, and U.S. publication 2009/0047719, filed Aug. 10, 2007.

Another computational method for identifying and designing metabolic alterations favoring biosynthetic production of a product is a metabolic modeling and simulation system termed SimPheny®. This computational method and system is described in, for example, U.S. publication 2003/0233218, filed Jun. 14, 2002, and in International Patent Application No. PCT/US03/18838, filed Jun. 13, 2003. SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components.

These computational approaches are consistent with biological realities because biological systems are flexible and can reach the same result in many different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which physiological performance or phenotype can be predicted.

Given the teachings and guidance provided herein, those skilled in the art will be able to apply various computational frameworks for metabolic modeling and simulation to design and implement biosynthesis of a desired compound in host microbial organisms. Such metabolic modeling and simulation methods include, for example, the computational systems exemplified above as SimPheny® and OptKnock. For illustration of the invention, some methods are described herein with reference to the OptKnock computation framework for modeling and simulation. Those skilled in the art will know how to apply the identification, design and implementation of the metabolic alterations using OptKnock to any of such other metabolic modeling and simulation computational frameworks and methods well known in the art.

The methods described above will provide one set of metabolic reactions to disrupt. Elimination of each reaction within the set or metabolic modification can result in a desired product as an obligatory product during the growth phase of the organism. Because the reactions are known, a solution to the bilevel OptKnock problem also will provide the associated gene or genes encoding one or more enzymes that catalyze each reaction within the set of reactions. Identification of a set of reactions and their corresponding genes encoding the enzymes participating in each reaction is generally an automated process, accomplished through correlation of the reactions with a reaction database having a relationship between enzymes and encoding genes.

Once identified, the set of reactions that are to be disrupted in order to achieve production of a desired product are implemented in the target cell or organism by functional disruption of at least one gene encoding each metabolic reaction within the set. One particularly useful means to achieve functional disruption of the reaction set is by deletion of each encoding gene. However, in some instances, it can be beneficial to disrupt the reaction by other genetic aberrations including, for example, mutation, deletion of regulatory regions such as promoters or cis binding sites for regulatory factors, or by truncation of the coding sequence at any of a number of locations. These latter aberrations, resulting in less than total deletion of the gene set can be useful, for example, when rapid assessments of the coupling of a product are desired or when genetic reversion is less likely to occur.

To identify additional productive solutions to the above described bilevel OptKnock problem which lead to further sets of reactions to disrupt or metabolic modifications that can result in the biosynthesis, including growth-coupled biosynthesis of a desired product, an optimization method, termed integer cuts, can be implemented. This method proceeds by iteratively solving the OptKnock problem exemplified above with the incorporation of an additional constraint referred to as an integer cut at each iteration. Integer cut constraints effectively prevent the solution procedure from choosing the exact same set of reactions identified in any previous iteration that obligatorily couples product biosynthesis to growth. For example, if a previously identified growth-coupled metabolic modification specifies reactions 1, 2, and 3 for disruption, then the following constraint prevents the same reactions from being simultaneously considered in subsequent solutions. The integer cut method is well known in the art and can be found described in, for example, Burgard et al., Biotechnol. Prog. 17:791-797 (2001). As with all methods described herein with reference to their use in combination with the OptKnock computational framework for metabolic modeling and simulation, the integer cut method of reducing redundancy in iterative computational analysis also can be applied with other computational frameworks well known in the art including, for example, SimPheny®.

The methods exemplified herein allow the construction of cells and organisms that biosynthetically produce a desired product, including the obligatory coupling of production of a target biochemical product to growth of the cell or organism engineered to harbor the identified genetic alterations. Therefore, the computational methods described herein allow the identification and implementation of metabolic modifications that are identified by an in silico method selected from OptKnock or SimPheny®. The set of metabolic modifications can include, for example, addition of one or more biosynthetic pathway enzymes and/or functional disruption of one or more metabolic reactions including, for example, disruption by gene deletion.

As discussed above, the OptKnock methodology was developed on the premise that mutant microbial networks can be evolved towards their computationally predicted maximum-growth phenotypes when subjected to long periods of growth selection. In other words, the approach leverages an organism's ability to self-optimize under selective pressures. The OptKnock framework allows for the exhaustive enumeration of gene deletion combinations that force a coupling between biochemical production and cell growth based on network stoichiometry. The identification of optimal gene/reaction knockouts requires the solution of a bilevel optimization problem that chooses the set of active reactions such that an optimal growth solution for the resulting network overproduces the biochemical of interest (Burgard et al., Biotechnol. Bioeng. 84:647-657 (2003)).

An in silico stoichiometric model of E. coli metabolism can be employed to identify essential genes for metabolic pathways as exemplified previously and described in, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379. As disclosed herein, the OptKnock mathematical framework can be applied to pinpoint gene deletions leading to the growth-coupled production of a desired product. Further, the solution of the bilevel OptKnock problem provides only one set of deletions. To enumerate all meaningful solutions, that is, all sets of knockouts leading to growth-coupled production formation, an optimization technique, termed integer cuts, can be implemented. This entails iteratively solving the OptKnock problem with the incorporation of an additional constraint referred to as an integer cut at each iteration, as discussed above.

As disclosed herein, a nucleic acid encoding a desired activity of an ethylene glycol pathway can be introduced into a host organism. In some cases, it can be desirable to modify an activity of an ethylene glycol pathway enzyme or protein to increase production of ethylene glycol. For example, known mutations that increase the activity of a protein or enzyme can be introduced into an encoding nucleic acid molecule. Additionally, optimization methods can be applied to increase the activity of an enzyme or protein and/or decrease an inhibitory activity, for example, decrease the activity of a negative regulator.

One such optimization method is directed evolution. Directed evolution is a powerful approach that involves the introduction of mutations targeted to a specific gene in order to improve and/or alter the properties of an enzyme. Improved and/or altered enzymes can be identified through the development and implementation of sensitive high-throughput screening assays that allow the automated screening of many enzyme variants (for example, >10⁴). Iterative rounds of mutagenesis and screening typically are performed to afford an enzyme with optimized properties. Computational algorithms that can help to identify areas of the gene for mutagenesis also have been developed and can significantly reduce the number of enzyme variants that need to be generated and screened. Numerous directed evolution technologies have been developed (for reviews, see Hibbert et al., Biomol. Eng 22:11-19 (2005); Huisman and Lalonde, In Biocatalysis in the pharmaceutical and biotechnology industries pgs. 717-742 (2007), Patel (ed.), CRC Press; Otten and Quax. Biomol. Eng 22:1-9 (2005).; and Sen et al., Appl Biochem. Biotechnol 143:212-223 (2007)) to be effective at creating diverse variant libraries, and these methods have been successfully applied to the improvement of a wide range of properties across many enzyme classes. Enzyme characteristics that have been improved and/or altered by directed evolution technologies include, for example: selectivity/specificity, for conversion of non-natural substrates; temperature stability, for robust high temperature processing; pH stability, for bioprocessing under lower or higher pH conditions; substrate or product tolerance, so that high product titers can be achieved; binding (Kₘ), including broadening substrate binding to include non-natural substrates; inhibition (Kᵢ), to remove inhibition by products, substrates, or key intermediates; activity (kcat), to increases enzymatic reaction rates to achieve desired flux; expression levels, to increase protein yields and overall pathway flux; oxygen stability, for operation of air sensitive enzymes under aerobic conditions; and anaerobic activity, for operation of an aerobic enzyme in the absence of oxygen.

A number of exemplary methods have been developed for the mutagenesis and diversification of genes to target desired properties of specific enzymes. Such methods are well known to those skilled in the art. Any of these can be used to alter and/or optimize the activity of an ethylene glycol pathway enzyme or protein. Such methods include, but are not limited to EpPCR, which introduces random point mutations by reducing the fidelity of DNA polymerase in PCR reactions (Pritchard et al., J Theor. Biol. 234:497-509 (2005)); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res. 32:e145 (2004); and Fujii et al., Nat. Protoc. 1:2493-2497 (2006)); DNA or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc Natl Acad Sci USA 91:10747-10751 (1994); and Stemmer, Nature 370:389-391 (1994)); Staggered Extension (StEP), which entails template priming followed by repeated cycles of 2 step PCR with denaturation and very short duration of annealing/extension (as short as 5 sec) (Zhao et al., Nat. Biotechnol. 16:258-261 (1998)); Random Priming Recombination (RPR), in which random sequence primers are used to generate many short DNA fragments complementary to different segments of the template (Shao et al., Nucleic Acids Res 26:681-683 (1998)).

Additional methods include Heteroduplex Recombination, in which linearized plasmid DNA is used to form heteroduplexes that are repaired by mismatch repair (Volkov et al, Nucleic Acids Res. 27:e18 (1999); and Volkov et al., Methods Enzymol. 328:456-463 (2000)); Random Chimeragenesis on Transient Templates (RACHITT), which employs Dnase I fragmentation and size fractionation of single stranded DNA (ssDNA) (Coco et al., Nat. Biotechnol. 19:354-359 (2001)); Recombined Extension on Truncated templates (RETT), which entails template switching of unidirectionally growing strands from primers in the presence of unidirectional ssDNA fragments used as a pool of templates (Lee et al., J. Molec. Catalysis 26:119-129 (2003)); Degenerate Oligonucleotide Gene Shuffling (DOGS), in which degenerate primers are used to control recombination between molecules; (Bergquist and Gibbs, Methods Mol. Biol. 352:191-204 (2007); Bergquist et al., Biomol. Eng 22:63-72 (2005); Gibbs et al., Gene 271:13-20 (2001)); Incremental Truncation for the Creation of Hybrid Enzymes (ITCHY), which creates a combinatorial library with 1 base pair deletions of a gene or gene fragment of interest (Ostermeier et al., Proc. Natl. Acad. Sci. USA 96:3562-3567 (1999); and Ostermeier et al., Nat. Biotechnol. 17:1205-1209 (1999)); Thio-Incremental Truncation for the Creation of Hybrid Enzymes (THIO-ITCHY), which is similar to ITCHY except that phosphothioate dNTPs are used to generate truncations (Lutz et al., Nucleic Acids Res 29:E16 (2001)); SCRATCHY, which combines two methods for recombining genes, ITCHY and DNA shuffling (Lutz et al., Proc. Natl. Acad. Sci. USA 98:11248-11253 (2001)); Random Drift Mutagenesis (RNDM), in which mutations made via epPCR are followed by screening/selection for those retaining usable activity (Bergquist et al., Biomol. Eng. 22:63-72 (2005)); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (Wong et al., Biotechnol. J. 3:74-82 (2008); Wong et al., Nucleic Acids Res. 32:e26 (2004); and Wong et al., Anal. Biochem. 341:187-189 (2005)); Synthetic Shuffling, which uses overlapping oligonucleotides designed to encode “all genetic diversity in targets” and allows a very high diversity for the shuffled progeny (Ness et al., Nat. Biotechnol. 20:1251-1255 (2002)); Nucleotide Exchange and Excision Technology NexT, which exploits a combination of dUTP incorporation followed by treatment with uracil DNA glycosylase and then piperidine to perform endpoint DNA fragmentation (Muller et al., Nucleic Acids Res. 33:e117 (2005)).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (Sieber et al., Nat. Biotechnol. 19:456-460 (2001)); Gene Site Saturation Mutagenesis™ (GSSM™), in which the starting materials include a supercoiled double stranded DNA (dsDNA) plasmid containing an insert and two primers which are degenerate at the desired site of mutations (Kretz et al., Methods Enzymol. 388:3-11 (2004)); Combinatorial Cassette Mutagenesis (CCM), which involves the use of short oligonucleotide cassettes to replace limited regions with a large number of possible amino acid sequence alterations (Reidhaar-Olson et al. Methods Enzymol. 208:564-586 (1991); and Reidhaar-Olson et al. Science 241:53-57 (1988)); Combinatorial Multiple Cassette Mutagenesis (CMCM), which is essentially similar to CCM and uses epPCR at high mutation rate to identify hot spots and hot regions and then extension by CMCM to cover a defined region of protein sequence space (Reetz et al., Angew. Chem. Int. Ed Engl. 40:3589-3591 (2001)); the Mutator Strains technique, in which conditional is mutator plasmids, utilizing the mutD5 gene, which encodes a mutant subunit of DNA polymerase III, to allow increases of 20 to 4000-X in random and natural mutation frequency during selection and block accumulation of deleterious mutations when selection is not required (Selifonova et al., Appl. Environ. Microbiol. 67:3645-3649 (2001)); Low et al., J. Mol. Biol. 260:359-3680 (1996)).

Additional exemplary methods include Look-Through Mutagenesis (LTM), which is a multidimensional mutagenesis method that assesses and optimizes combinatorial mutations of selected amino acids (Rajpal et al., Proc. Natl. Acad. Sci. USA 102:8466-8471 (2005)); Gene Reassembly, which is a DNA shuffling method that can be applied to multiple genes at one time or to create a large library of chimeras (multiple mutations) of a single gene (Tunable GeneReassembly™ (TGR™) Technology supplied by Verenium Corporation), in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (Hayes et al., Proc. Natl. Acad. Sci. USA 99:15926-15931 (2002)); and Iterative Saturation Mutagenesis (ISM), which involves using knowledge of structure/function to choose a likely site for enzyme improvement, performing saturation mutagenesis at chosen site using a mutagenesis method such as Stratagene QuikChange (Stratagene; San Diego Calif.), screening/selecting for desired properties, and, using improved clone(s), starting over at another site and continue repeating until a desired activity is achieved (Reetz et al., Nat. Protoc. 2:891-903 (2007); and Reetz et al., Angew. Chem. Int. Ed Engl. 45:7745-7751 (2006)).

Any of the aforementioned methods for mutagenesis can be used alone or in any combination. Additionally, any one or combination of the directed evolution methods can be used in conjunction with adaptive evolution techniques, as described herein.

It is understood that modifications which do not substantially affect the activity of the various embodiments of this invention are also provided within the definition of the invention provided herein. Accordingly, the following examples are intended to illustrate but not limit the present invention.

### Example I

### Pathways for Producing Ethylene Glycol from Serine

Several pathways are shown in FIG. 1 for synthesis of MEG from serine. In one embodiment serine is converted to hydroxypyruvate by a serine-hydroxypyruvate aminotransferase or a serine oxidoreductase (deaminating) (FIG. 1, Steps 1 or 2). Hydroxypyruvate is subsequently decarboxylated to glycoloaldehyde by hydroxypyruvate decarboxylase (FIG. 1, Step 3). Finally, glycolaldehyde is reduced to MEG by an aldehyde reductase (FIG. 1, Step 4). In an alternate route, the hydroxypyruvate intermediate is reduced to glycerate by hydroxypyruvate reductase, and subsequently decarboxylated yielding ethylene glycol (FIG. 1, Steps 8 and 9). In yet another pathway, serine is first decarboxylated to ethanolamine (FIG. 1, Step 5). This compound is subsequently converted to glycolaldehyde by a serine aminotransferase or oxidoreductase (deaminating) (FIG. 1, Steps 6 or 7). Exemplary enzyme candidates for serine pathway enzymes (Steps 1-9 of FIG. 1) are described below.

The conversion of serine to hydroxypyruvate (FIG. 1, Step 1) is catalyzed by an enzyme with serine aminotransferase activity. Exemplary enzymes include serine:pyruvate aminotransferase (EC 2.6.1.510), alanine:glyoxylate aminotransferase (EC 2.6.1.44) and serine: glyoxylate aminotransferase (EC 2.6.1.45). Serine:pyruvate aminotransferase participates in serine metabolism and glyoxylate detoxification in mammals. These enzymes have been shown to utilize a variety of alternate oxo donors such as pyruvate, phenylpyruvate and glyoxylate; and amino acceptors including alanine, glycine and phenylalanine (Ichiyama et al., Mol. Urol. 4:333-340 (2000)). The rat mitochondria serine:pyruvate aminotransferase, encoded by agxt, is also active as an alanine-glyoxylate aminotransferase. This enzyme was heterologously expressed in E. coli (Oda et al., J Biochem. 106:460-467 (1989)). Similar enzymes have been characterized in humans and flies (Oda et al., Biochem. Biophys. Res. Commun. 228:341-346 (1996)). The human enzyme, encoded by agxt, functions as a serine:pyruvate aminotransferase, an alanine: glyoxylate aminotransferase and a serine:glyoxylate aminotransferase (Nagata et al., Biomed. Res. 30:295-301 (2009)). The fly enzyme is encoded by spat (Han et al., FEBS Lett. 527:199-204 (2002)). An exemplary alanine:glyoxylate aminotransferases is encoded by AGT1 of Arabidopsis thaliana. In addition to the alanine:glyoxylate acitivty, the purified, recombinant AGT1 expressed in E. coli also catalyzed serine:glyoxylate and serine:pyruvate aminotransferase activities (Liepman et al., Plant J 25:487-498 (2001)). In several organisms serine:glyoxylate aminotransferase enzymes (EC 2.6.1.45) also exhibit reduced but detectable serine:pyruvate aminotransferase activity. Exemplary enzymes are found in Phaseolus vulgaris, Pisum sativum, Secale cereal and Spinacia oleracea. Serine:glyoxylate aminotransferase enzymes interconvert serine and hydroxypyruvate and utilize glyoxylate as an amino acceptor. The serine:glyoxylate aminotransferase from the obligate methylotroph Hyphomicrobium methylovorum GM2 has been functionally expressed in E. coli and characterized (Hagishita et al., Eur. J Biochem. 241:1-5 (1996)).

| Protein | GenBank ID | GI Number | Organism |
|---|---|---|---|
| Agxt | NP_085914.1 | 13470096 | Rattus norvegicus |
| Agxt | NP_085914.1 | 13470096 | Rattus norvegicus |
| Agxt | NP_000021.1 | 4557289 | Homo sapiens |
| Spat | NP_511062.1 | 17530823 | Drosophila |
| | | | melanogaster |
| AGT1 | NP_849951.1 | 30678921 | Arabidopsis |
| | | | thaliana |
| D86125.1:914..2131 | BAA19919.1 | 2081618 | Hyphomicrobium |
| | | | methylovorum |

The conversion of serine to hydroxypyruvate (FIG. 1, Step 1) is alternately catalyzed by serine oxidoreductase (deaminating). One enzyme with this functionality is serine oxidase, which utilizes oxygen as an electron acceptor, converting serine, O₂ and water to ammonia, hydrogen peroxide and hydroxypyruvate (Chumakov, et al., Proc. Nat. Acad. Sci., 99(21):13675-13680; Verral et al., Eur J. Neurosci., 26(6) 1657-1669 (2007)). Some amino oxidases are specific for the D-amino acid (Dixon and Kleppe, Biochim Biophys Acta, 96: 368-382 (1965)) and L-serine can be converted to D-serine by serine racemace (Miranda, et al., Gene, 256:183-188 (2000)). Enzymes in the EC class 1.4.1 catalyze the oxidative deamination of alpha-amino acids with NAD+, NADP+ or FAD as acceptor, and the reactions are typically reversible. Exemplary enzymes with serine oxidoreductase (deaminating) activity include serine dehydrogenase (EC 1.4.1.7), L-amino acid dehydrogenase (EC 1.4.1.5) and glutamate dehydrogenase (EC 1.4.1.2). An enzyme with serine dehydrogenase activity from Petroselinum crispum was purified and characterized although the gene associated with the enzyme has not been identified to date (Kretovich et al., Izv. Akad. Nauk SSSR Ser. Biol. 2:295-301 (1966)). Serine dehydrogenase activity attributed to L-amino-acid dehydrogenase was identified in soil bacteria isolates, but specific genes were not identified (Mohammadi et al., Iran Biomed. J 11:131-135 (2007)). The glutamate dehydrogenase from Vigna unguiculata accepts serine as an alternate substrate. The gene associated with this enzyme has not been identified to date. Other glutamate dehydrogenase enzymes are encoded by gdhA in Escherichia coli (Korber et al., J Mol. Biol. 234:1270-1273 (1993); McPherson et al., Nucleic Acids Res. 11:5257-5266 (1983)), gdh from Thermotoga maritime (Kort et al., Extremophiles. 1:52-60 (1997); Lebbink et al., J Mol. Biol. 280:287-296 (1998); Lebbink et al., J Mol. Biol. 289:357-369 (1999)), and gdhA1 from Halobacterium salinarum (Ingoldsby et al., Gene 349:237-244 (2005)).

| Protein | GenBank ID | GI Number | Organism |
|---|---|---|---|
| gdhA | 118547 | P00370 | Escherichia coli |
| gdh | 6226595 | P96110.4 | Thermotoga maritima |
| gdhA1 | 15789827 | NP_279651.1 | Halobacterium salinarum |

Decarboxylation of hydroxypyruvate to glycolaldehyde (FIG. 1, Step 3 and FIG. 2, Step 3) is catalyzed by hydroxypyruvate decarboxylase (EC 4.1.1.40), an enzyme found in many mammals (Hendrick et al., Arch. Biochem. Biophys. 105:261-269 (1964)). The enzyme activity has been studied in the context of hydroxypyruvate metabolism to oxalate in rat mitochondria, although the activity is not associated with a gene to date (Rofe et al., Biochem. Med. Metab Biol. 36:141-150 (1986)). Other keto-acid decarboxylases include pyruvate decarboxylase (EC 4.1.1.1), benzoylformate decarboxylase (EC 4.1.1.7), alpha-ketoglutarate decarboxylase and branched-chain alpha-ketoacid decarboxylase. Several keto-acid decarboxylase enzymes have been shown to accept hydroxypyruvate as an alternate substrate, including the kivd gene product of Lactococcus lactis (de la Plaza et al., FEMS Microbiol Lett. 238:367-374 (2004)) and the pdc1 gene product of Saccharomyces cerevisiae (Cusa et al., J Bacteriol. 181:7479-7484 (1999)). The S. cerevisiae enzyme has been extensively studied, engineered for altered activity, and functionally expressed in E. coli (Killenberg-Jabs et al., Eur. J. Biochem. 268:1698-1704 (2001); Li et al., Biochemistry. 38:10004-10012 (1999); ter Schure et al., Appl. Environ. Microbiol. 64:1303-1307 (1998)). The PDC from Zymomonas mobilus, encoded by pdc, also has a broad substrate range and has been a subject of directed engineering studies to alter the affinity for different substrates (Siegert et al., Protein Eng Des Sel 18:345-357 (2005)). An additional candidate is the kdcA gene product of Lactococcus lactis, which decarboxylates a variety of branched and linear ketoacid substrates including 2-oxobutanoate, 2-oxohexanoate, 2-oxopentanoate, 3-methyl-2-oxobutanoate, 4-methyl-2-oxobutanoate and isocaproate (Smit et al., Appl Environ Microbiol 71:303-311 (2005)).

| Protein | GenBank ID | GI Number | Organism |
|---|---|---|---|
| kivd | CAG34226.1 | 51870502 | Lactococcus lactis |
| pdc1 | P06169 | 30923172 | Saccharomyces cerevisiae |
| pdc | P06672.1 | 118391 | Zymomonas mobilis |
| kdcA | AAS49166.1 | 44921617 | Lactococcus lactis |

The reduction of glycolaldehyde to ethylene glycol (all Figures) is catalyzed by glycolaldehyde reductase. The iron-activated 1,2-PDO oxidoreductase (EC 1.1.1.77) E. coli encoded by fucO efficiently catalyzes the reduction of glycolaldehyde (Obradors et al., Eur. J. Biochem. 258:207-213 (1998); Boronat et al., J Bacteriol. 153:134-139 (1983)). Other aldehyde reductase enzyme candidates include alrA from Acinetobacter sp. Strain M-1 encoding a medium-chain alcohol dehydrogenase for C2-C14 (Tani et al., Appl. Environ. Microbiol. 66:5231-5235 (2000)), ADH2 from Saccharomyces cerevisiae (Atsumi et al., Nature 451:86-89 (2008)) and the adhA gene product from Zymomonas mobilis, which was demonstrated to have activity on a number of aldehydes including formaldehyde, acetaldehyde, propionaldehyde, butyraldehyde, and acrolein (Kinoshita et al., Appl Microbiol Biotechnol 22:249-254 (1985)).

| Protein | GenBank ID | GI Number | Organism |
|---|---|---|---|
| fucO | AAA23825.1 | 146045 | Escherichia coli |
| alrA | BAB12273.1 | 9967138 | Acinetobacter sp. Strain M-1 |
| ADH2 | NP_014032.1 | 6323961 | Saccharomyces cerevisiae |
| adhA | YP_162971.1 | 56552132 | Zymomonas mobilis |

Serine decarboxylase (EC 4.1.1.-) catalyzes the decarboxylation of serine to ethanolamine (FIG. 1, Step 5). Enzymes with this activity have been characterized in plants such as Spinacia oleracea, Arabidopsis thaliana and Brassica napus in the context of choline biosynthesis. The A. thaliana serine decarboxylase encoded by AtSDC is a soluble tetramer and was characterized by heterologous expression in E. coli and ability to complement a yeast mutant deficient in ethanolamine biosynthesis (Rontein et al., J Biol. Chem. 276:35523-35529 (2001)). The Brassica napus serine decarboxylase was identified and characterized in the same study. A similar enzyme is found in Spinacia oleracea although the gene has not been identified to date (Summers et al., Plant Physiol 103:1269-1276 (1993)). Other serine decarboxylase candidates can be identified by sequence homology to the Arabidopsis or Brassica enzymes. A candidate with high homology is the putative serine decarboxylase from Beta vulgaris.

| Protein | GenBank ID | GI Number | Organism |
|---|---|---|---|
| AtSDC | AAK77493.1 | 15011302 | Arabidopsis thaliana |
| BnSDC | BAA78331.1 | 4996105 | Brassica napus |
| BvSDC1 | BAE07183.1 | 71000475 | Beta vulgaris |

The conversion of ethanolamine to glycolaldehyde is catalyzed by an enzyme with ethanolamine aminotransferase activity. Such an enzyme activity has not been demonstrated to date. Exemplary candidates are aminotransferases with broad substrate specificity that convert terminal amines to aldehydes, such as gamma-aminobutyrate GABA transaminase (EC 2.6.1.19), diamine aminotransferase (EC 2.6.1.29) and putrescine aminotransferase (EC 2.6.1.82). GABA aminotransferase naturally interconverts succinic semialdehyde and glutamate to 4-aminobutyrate and alpha-ketoglutarate and is known to have a broad substrate range (Schulz et al., 56:1-6 (1990); Liu et al., 43:10896-10905 (2004)). The two GABA transaminases in E. coli are encoded by gabT (Bartsch et al., J Bacteriol. 172:7035-7042 (1990)) and puuE (Kurihara et al., J. Biol. Chem. 280:4602-4608 (2005)). GABA transaminases in Mus musculus and Sus scrofa have also been shown to react with a range of alternate substrates (Cooper, Methods Enzymol. 113:80-82 (1985)). Additional enzyme candidates for interconverting ethanolamine and glycolaldehyde are putrescine aminotransferases and other diamine aminotransferases. The E. coli putrescine aminotransferase is encoded by the ygjG gene and the purified enzyme also was able to transaminate cadaverine and spermidine (Samsonova et al., BMC. Microbiol 3:2 (2003)). In addition, activity of this enzyme on 1,7-diaminoheptane and with amino acceptors other than 2-oxoglutarate (e.g., pyruvate, 2-oxobutanoate) has been reported (Samsonova et al., BMC. Microbiol 3:2 (2003); Kim, J Biol. Chem. 239:783-786 (1964)).

| Protein | GenBank ID | GI Number | Organism |
|---|---|---|---|
| gabT | NP_417148.1 | 16130576 | Escherichia coli |
| puuE | NP_415818.1 | 16129263 | Escherichia coli |
| abat | NP_766549.2 | 37202121 | Mus musculus |
| abat | NP_999428.1 | 47523600 | Sus scrofa |
| ygjG | NP_417544 | 145698310 | Escherichia coli |

The oxidative deamination of ethanolamine to glycolaldehyde is catalyzed by ethanolamine oxidoreductase (deaminating). One enzyme with this functionality is ethanolamine oxidase (EC 1.4.3.8), which utilizes oxygen as an electron acceptor, converting ethanolamine, O₂ and water to ammonia, hydrogen peroxide and glycolaldehyde (Schomburg et al., Springer Handbook of Enzymes. 320-323 (2005)). Ethanolamine oxidase has been characterized in Pseudomonas sp and Phormia regina; however, the enzyme activity has not been associated with a gene to date. Alternately, the oxidative deamination of ethanolamine can be catalyzed by a deaminating oxidoreductase that utilizes NAD+, NADP+ or FAD as acceptor. An exemplary enzyme for catalyzing the conversion of a primary amine to an aldehyde is lysine 6-dehydrogenase (EC 1.4.1.18), encoded by the lysDH genes. This enzyme catalyzes the oxidative deamination of the 6-amino group of L-lysine to form 2-aminoadipate-6-semialdehyde (Misono et al., J Bacteriol. 150:398-401 (1982)). Additional enzyme candidates are found in Geobacillus stearothermophilus (Heydari et al., Appl Environ. Microbiol 70:937-942 (2004)), Agrobacterium tumefaciens (Hashimoto et al., J Biochem. 106:76-80 (1989); Misono and Nagasaki, J Bacteriol. 150:398-401 (1982)), and Achromobacter denitrificans (Ruldeekulthamrong et al., BMB. Rep. 41:790-795 (2008)).

| Protein | GenBank ID | GI Number | Organism |
|---|---|---|---|
| lysDH | BAB39707 | 13429872 | Geobacillus stearothermophilus |
| lysDH | NP_353966 | 15888285 | Agrobacterium tumefaciens |
| lysDH | AAZ94428 | 74026644 | Achromobacter denitrificans |

Hydroxypyruvate reductase (EC 1.1.1.29 and EC 1.1.1.81), also called glycerate dehydrogenase, catalyzes the reversible NAD(P)H-dependent reduction of hydroxypyruvate to glycerate (FIG. 1, Step 8). The ghrA and ghrB genes of E. coli encode enzymes with hydroxypyruvate reductase activity (Nunez et al., Biochem. J 354:707-715 (2001)). Both gene products also catalyze the reduction of glyoxylate to glycolate and the ghrB gene product prefers hydroxypyruvate as a substrate. Hydroxypyruvate reductase participates in the serine cycle in methylotrophic bacterium such as Methylobacterium extorquens AM1 and Hyphomicrobium methylovorum (Chistoserdova et al., J Bacteriol. 185:2980-2987 (2003)). Hydroxypyruvate reductase enzymes from Hyphomicrobium methylovorum and Methylobacterium sp. MB200 have been cloned and heterologously expressed in E. coli (Yoshida et al., Eur. J Biochem. 223:727-732 (1994)). The Methylobacterium sp. MB200 HPR has not been assigned a GenBank identifier to date but the sequence is available in the literature and bears 98% identity to the sequence of the M. extorquens hprA gene product, which uses both NADH and NADPH as cofactors (Chistoserdova et al., J Bacteriol. 173:7228-7232 (1991)). Bifunctional enzymes with hydroxypyruvate reductase and glyoxylate reductase activities (GRHPR) are found in mammals including Homo sapiens and Mus musculus. Recombinant NADPH-dependent GRHPR enzymes from these organisms were heterologously expressed in E. coli (Booth et al., J Mol. Biol. 360:178-189 (2006)).

| Protein | GenBank ID | GI Number | Organism |
|---|---|---|---|
| ghrA | NP_415551.2 | 90111205 | Escherichia coli |
| ghrB | NP_418009.2 | 90111614 | Escherichia coli |
| D31857.1:286..1254 | BAA06662.1 | 1304133 | Hyphomicrobium |
| | | | methylovorum |
| hprA | ACS39571.1 | 240008345 | Methylobacterium |
| | | | extorquens |
| GRHPR | NP_036335.1 | 6912396 | Homo sapiens |
| GRHPR | NP_525028.1 | 17933768 | Mus musculus |

An enzyme with glycerate decarboxylase activity is required to convert glycerate to ethylene glycol (FIG. 1, Step 9). Such an enzyme has not been characterized to date. However, a similar alpha,beta-hydroxyacid decarboxylation reaction is catalyzed by tartrate decarboxylase (EC 4.1.1.73). The enzyme, characterized in Pseudomonas sp. group Ve-2, is NAD⁺ dependent and catalyzes a coupled oxidation-reduction reaction that proceeds through an oxaloglycolate intermediate (Furuyoshi et al., J Biochem. 110:520-525 (1991)). A side reaction catalyzed by this enzyme is the NAD⁺ dependent oxidation of tartrate (1% of activity). Glycerate was not reactive as a substrate for this enzyme and was instead an inhibitor, so enzyme engineering or directed evolution is likely required for this enzyme to function in the desired context. A gene has not been associated with this enzyme activity to date.

An additional candidate glycerate decarboxylase is acetolactate decarboxylase (EC 4.1.1.5) which participates in citrate catabolism and branched-chain amino acid biosynthesis, converting the 2-hydroxyacid 2-acetolactate to acetoin. In Lactococcus lactis the enzyme is a hexamer encoded by gene aldB, and is activated by valine, leucine and isoleucine (Goupil-Feuillerat et al., J. Bacteriol. 182:5399-5408 (2000); Goupil et al., Appl. Environ. Microbiol. 62:2636-2640 (1996)). This enzyme has been overexpressed and characterized in E. coli (Phalip et al., FEBS Lett. 351:95-99 (1994)). In other organisms the enzyme is a dimer, encoded by aldC in Streptococcus thermophilus (Monnet et al., Lett. Appl. Microbiol. 36:399-405 (2003)), aldB in Bacillus brevis (Najmudin et al., Acta Crystallogr. D. Biol. Crystallogr. 59:1073-1075 (2003); Diderichsen et al., J. Bacteriol. 172:4315-4321 (1990)) and budA from Enterobacter aerogenes (Diderichsen et al., J. Bacteriol. 172:4315-4321 (1990)). The enzyme from Bacillus brevis was cloned and overexpressed in Bacillus subtilis and structurally characterized (Najmudin et al., Acta Crystallogr. D. Biol. Crystallogr. 59:1073-1075 (2003)). A similar enzyme from Leuconostoc lactis has been purified and characterized but the gene has not been isolated to date (O'Sullivan et al., FEMS Microbiol. Lett. 194:245-249 (2001)).

| Protein | GenBank ID | GI Number | Organism |
|---|---|---|---|
| aldB | NP_267384.1 | 15673210 | Lactococcus lactis |
| aldC | Q8L208 | 75401480 | Streptococcus thermophilus |
| aldB | P23616.1 | 113592 | Bacillus brevis |
| budA | P05361.1 | 113593 | Enterobacter aerogenes |

### Example II

### Pathways for producing ethylene glycol from 3-phosphoglycerate

Also shown in FIG. 1 are pathways to convert 3-phosphoglycerate (3PG) to ethylene glycol. In these pathways, 3-phosphoglycerate is first converted to glycerate by either a 3PG phosphatase or a glycerate kinase enzyme operating in the glycerate-generating direction (FIG. 1, Steps 10 or 11). Glycerate is then directly decarboxylated to ethylene glycol (FIG. 1, Step 9). Alternately, glycerate is oxidized to hydroxypyruvate (FIG. 1, Step 8), which is subsequently converted to ethylene glycol by the combined actions of hydroxypyruvate decarboxylase and glycolaldehyde reductase as described previously. Enzyme candidates for steps 10-11 of FIG. 1 are provided below.

3-Phosphoglycerate phosphatase (EC 3.1.3.38) catalyzes the hydrolysis of 3PG to glycerate, releasing pyrophosphate (FIG. 1, Step 10). The enzyme is found in plants and has a broad substrate range that includes phosphoenolpyruvate, ribulose-1,5-bisphosphate, dihydroxyacetone phosphate and glucose-6-phosphate (Randall et al., Plant Physiol 48:488-492 (1971); Randall et al., J Biol. Chem. 246:5510-5517 (1971)). Purified enzyme from various plant sources has been characterized but a gene has not been associated with this enzyme to date. Another enzyme with 3-phosphoglycerate phosphatase activity is the phosphoglycerate phosphatase (EC 3.1.3.20) from pig liver (Fallon et al., Biochim. Biophys. Acta 105:43-53 (1965)). The gene associated with this enzyme is not available.

The enzyme alkaline phosphatase (EC 3.1.3.1) hydrolyses a broad range of phosphorylated substrates to their corresponding alcohols. These enzymes are typically secreted into the periplasm in bacteria, where they play a role in phosphate transport and metabolism. The E. coli phoA gene encodes a periplasmic zinc-dependent alkaline phosphatase active under conditions of phosphate starvation (Coleman Annu. Rev. Biophys. Biomol. Struct. 21:441-83 (1992)). Similar enzymes have been characterized in Campylobacter jejuni (van Mourik et al., Microbiol. 154:584-92 (2008)), Saccharomyces cerevisiae (Oshima et al., Gene 179:171-7 (1996)) and Staphylococcus aureus (Shah and Blobel, J. Bacteriol. 94:780-1 (1967)). Enzyme engineering and/or removal of targeting sequences may be required for alkaline phosphatase enzymes to function in the cytoplasm.

| Protein | GenBank ID | GI Number | Organism |
|---|---|---|---|
| phoA | NP_414917.2 | 49176017 | Escherichia coli |
| phoX | ZP_01072054.1 | 86153851 | Campylobacter jejuni |
| PHO8 | AAA34871.1 | 172164 | Saccharomyces |
| | | | cerevisiae |
| SaurJH1_2706 | YP_001317815.1 | 150395140 | Staphylococcus aureus |

The interconversion of 3-phosphoglycerate and glycerate (FIG. 1, Step 11) is also catalyzed by glycerate kinase (EC 2.7.1.31). This enzyme naturally operates in the ATP-consuming phosphorylation direction and has not been shown to function in the ATP-generating direction. Three classes of glycerate kinase have been identified. Enzymes in class I and II produce glycerate-2-phosphate, whereas the class III enzymes found in plants and yeast produce glycerate-3-phosphate (Bartsch et al., FEBS Lett. 582:3025-3028 (2008)). In a recent study, class III glycerate kinase enzymes from Saccharomyces cerevisiae, Oryza sativa and Arabidopsis thaliana were heterologously expressed in E. coli and characterized (Bartsch et al., FEBS Lett. 582:3025-3028 (2008)). This study also assayed the glxK gene product of E. coli for ability to form glycerate-3-phosphate and found that the enzyme can only catalyze the formation of glycerate-2-phosphate, in contrast to previous work (Doughty et al., J Biol. Chem. 241:568-572 (1966)).

| Protein | GenBank ID | GI Number | Organism |
|---|---|---|---|
| glxK | AAC73616.1 | 1786724 | Escherichia coli |
| YGR205W | AAS56599.1 | 45270436 | Saccharomyces cerevisiae |
| Os01g0682500 | BAF05800.1 | 113533417 | Oryza sativa |
| At1g80380 | BAH57057.1 | 227204411 | Arabidopsis thaliana |

### Example III

### Pathways for Producing Ethylene Glycol from Glyoxylate Via Tartronate Semialdehyde

FIG. 2 shows a pathway for producing ethylene glycol from glyoxylate via a tartrate semialdehyde intermediate. The glyoxylate precursor may be derived from central metabolites such as isocitrate, via isocitrate lyase, or glycine, via one of several aminotransferase enzymes that utilize glycine as an amino donor such as serine:glyoxylate aminotransferase or glycine aminotransferase. In the proposed pathway, two equivalents of glyoxylate are joined by glyoxylate carboligase to form one equivalent of tartronate semialdehyde (FIG. 2, Step 1). Tartronate semialdehyde is subsequently isomerized to form hydroxypyruvate by hydroxypyruvate isomerase (FIG. 2, Step 2). The decarboxylation and reduction of hydroxypyruvate yield ethylene glycol as described previously (FIG. 2, Steps 3 and 4). Enzyme candidates for steps 1 and 2 of FIG. 2 are provided below.

Glyoxylate carboligase (EC 4.1.1.47), also known as tartrate semialdehyde synthase, catalyzes the condensation of two molecules of glyoxylate to form tartronate semialdehyde (FIG. 2, Step 2). The E. coli enzyme, encoded by gcl, is active under anaerobic conditions and requires FAD for activity although no net redox reaction takes place (Chang et al., J Biol. Chem. 268:3911-3919 (1993)). Glyoxylate carboligase activity has also been detected in Ralstonia eutropha, where it is encoded by h16_A3598 (Eschmann et al., Arch. Microbiol. 125:29-34 (1980)). Additional candidate glyoxylate carboligase enzyme candidates can be identified by sequence homology. Two exemplary candidates with high homology to the E. coli enzyme are found in Salmonella enterica and Burkholderia ambifaria.

| Protein | GenBank ID | GI Number | Organism |
|---|---|---|---|
| gcl | AAC73609.1 | 1786717 | Escherichia coli |
| h16_A3598 | YP_728024.1 | 113869535 | Ralstonia eutropha |
| SentesTy_010100014274 | ZP_03378393.1 | 213648340 | Salmonella enterica |
| Bamb_1815 | YP_773705.1 | 115351866 | Burkholderia ambifaria |

Hydroxypyruvate isomerase catalyzes the reversible isomerization of hydroxypyruvate and tartronate semialdehyde. The E. coli enzyme, encoded by hyi, is cotranscribed with glyoxylate carboligase (gcl) in a glyoxylate utilization operon (Ashiuchi et al., Biochim. Biophys. Acta 1435:153-159 (1999); Cusa et al., J Bacteriol. 181:7479-7484 (1999)). This enzyme has also been purified and characterized in Bacillus fastidiosus, although the associated gene is not known (de Windt et al., Biochim. Biophys. Acta 613:556-562 (1980)). Hydroxypyruvate isomerase enzyme candidates in other organisms such as Ralstonia eutropha and Burkholderia ambifaria can be identified by sequence homology to the E. coli gene product. Note that the predicted hydroxypyruvate isomerase gene candidates in these organisms are also co-localized with genes predicted to encode glyoxylate carboligase.

| Protein | GenBank ID | GI Number | Organism |
|---|---|---|---|
| hyi | AAC73610.1 | 1786718 | Escherichia coli |
| h16_A3599 | YP_728025.1 | 113869536 | Ralstonia eutropha |
| Bamb_1816 | YP_773706.1 | 115351867 | Burkholderia ambifaria |

### Example IV

### Pathways for Producing Ethylene Glycol from Glyoxylate Via Glycolate

Additional pathways from glyoxylate to ethylene glycol proceed through the intermediate glycolate as shown in FIG. 3. In the first step of all pathways, glyoxylate is converted to glycolate by glyoxylate reductase (FIG. 3, Step 1). Glycolate is then converted to ethylene glycol by one of several routes. In one route, glycolate is converted to glycolyl-CoA by a CoA transferase or synthetase (FIG. 3, Step 2/3). Glycolyl-CoA is then reductively deacylated to glycolaldehyde by glycolyl-CoA reductase (aldehyde forming) (FIG. 3, Step 4). Glycolaldehyde is converted to ethylene glycol by glycolaldehyde reductase as described previously (FIG. 3, Step 5). Alternately, glycolyl-CoA is directly converted to ethylene glycol by a bifunctional enzyme with CoA reductase (alcohol forming) activity (FIG. 3, Step 10). In an alternative route, glycolate is directly converted to glycolaldehyde by a carboxylic acid reductase enzyme with glycolate reductase activity (FIG. 3, Step 6). In yet another route, glycolate is converted to glycolaldehyde via a glycolylphosphate intermediate by the enzymes glycolate kinase and glycolylphosphate reductase (FIG. 3, Steps 7 and 9). Alternatively, the glycolylphosphate intermediate is converted to glycolyl-CoA by phosphotransglycolylase (FIG. 3, Step 8). Enzyme candidates for each of these steps are provided below.

The reduction of glyoxylate to glycolate is catalyzed by glyoxylate reductase (EC 1.1.1.79 and EC 1.1.1.26). In E. coli this reaction is catalyzed by the products of two genes, ghrB and ghrA (Nunez et al., Biochem. J 354:707-715 (2001)). Both gene products utilize NADPH and also catalyze the reduction of hydroxypyruvate and the ghrA gene product prefers glycolate as a substrate. Eukaryotic glyoxylate reductase enzyme candidates that have been expressed in E. coli include the NADPH or NADH dependent YNL274C gene product from S. cerevisiae (Rintala et al., Yeast 24:129-136 (2007)) and GR1 of Arabidopsis thaliana (Hoover et al., Can. J. Bot. 85:896-902 (2007); Allan et al., J Exp. Bot. 59:2555-2564 (2008)). The yeast enzyme also catalyzes the reduction of hydroxypyruvate.

| Protein | GenBank ID | GI Number | Organism |
|---|---|---|---|
| ghrA | NP_415551.2 | 90111205 | Escherichia coli |
| ghrB | NP_418009.2 | 90111614 | Escherichia coli |
| YNL274C | AAT92679.1 | 51012771 | Saccharomyces cerevisiae |
| GR1 | NP_566768.1 | 18404556 | Arabidopsis thaliana |

The activation of glycolate to glycolyl-CoA is catalyzed by an enzyme with glycolyl-CoA transferase activity. Such an enzyme has not been characterized to date. Glutaconyl-CoA transferase (EC 2.8.3.12) catalyzes the transfer of the 2-hydroxyacid, 2-hydroxyglutarate, to CoA. The glutaconyl-CoA-transferase (EC 2.8.3.12) enzyme from anaerobic bacterium Acidaminococcus fermentans reacts with a range of substrates including 2-hydroxyglutarate, glutarate, crotonate, adipate and acrylate (Buckel et al., Eur. J Biochem. 118:315-321 (1981); Mack et al., Eur. J. Biochem. 226:41-51 (1994)). The genes encoding this enzyme, gctA and gctB, have been cloned and functionally expressed in E. coli (Mack et al., supra).

| Protein | GenBank ID | GI Number | Organism |
|---|---|---|---|
| gctA | 559392 | CAA57199.1 | Acidaminococcus fermentans |
| gctB | 559393 | CAA57200.1 | Acidaminococcus fermentans |

The ATP-dependent acylation of glycolylate to glycolyl-CoA (FIG. 3, Step 3) is catalyzed by a glycolyl-CoA synthetase or acid-thiol ligase. Enzymes catalyzing this exact transformation have not been characterized to date; however, several enzymes with broad substrate specificities have been described in the literature. ADP-forming acetyl-CoA synthetase (ACD, EC 6.2.1.13) is an enzyme that couples the conversion of acids to their corresponding acyl-CoA esters with the concomitant consumption of ATP. ACD I from Archaeoglobus fulgidus, encoded by AF1211, was shown to operate on a variety of linear and branched-chain substrates including isobutyrate, isopentanoate, and fumarate (Musfeldt et al., J Bacteriol. 184:636-644 (2002)). A second reversible ACD in Archaeoglobus fulgidus, encoded by AF1983, was also shown to have a broad substrate range with high activity on cyclic compounds phenylacetate and indoleacetate (Musfeldt et al., supra). The enzyme from Haloarcula marismortui, annotated as a succinyl-CoA synthetase, accepts propionate, butyrate, and branched-chain acids (isovalerate and isobutyrate) as substrates, and was shown to operate in the forward and reverse directions (Brasen et al., Arch. Microbiol 182:277-287 (2004)). The ACD encoded by PAE3250 from hyperthermophilic crenarchaeon Pyrobaculum aerophilum showed the broadest substrate range of all characterized ACDs, reacting with acetyl-CoA, isobutyryl-CoA (preferred substrate) and phenylacetyl-CoA (Brasen and Schonheit, Arch. Microbiol 182:277-287 (2004)). Directed evolution or engineering can be used to modify this enzyme to operate at the physiological temperature of the host organism. The enzymes from A. fulgidus, H. marismortui and P. aerophilum have all been cloned, functionally expressed, and characterized in E. coli (Brasen and Schonheit, Arch. Microbiol 182:277-287 (2004); Musfeldt and Schonheit, J. Bacteriol. 184:636-644 (2002)). An additional candidate is the enzyme encoded by sucCD in E. coli, which naturally catalyzes the formation of succinyl-CoA from succinate with the concomitant consumption of one ATP, a reaction which is reversible in vivo (Buck et al., Biochemistry 24:6245-6252 (1985)). The acyl CoA ligase from Pseudomonas putida has been demonstrated to work on several aliphatic substrates including acetic, propionic, butyric, valeric, hexanoic, heptanoic, and octanoic acids and on aromatic compounds such as phenylacetic and phenoxyacetic acids (Fernandez-Valverde et al., Appl. Environ. Microbiol. 59:1149-1154 (1993)). A related enzyme, malonyl CoA synthetase (6.3.4.9) from Rhizobium leguminosarum could convert several diacids, namely, ethyl-, propyl-, allyl-, isopropyl-, dimethyl-, cyclopropyl-, cyclopropylmethylene-, cyclobutyl-, and benzyl-malonate into their corresponding monothioesters (Pohl et al., J. Am. Chem. Soc. 123:5822-5823 (2001)).

| Protein | GenBank ID | GI Number | Organism |
|---|---|---|---|
| AF1211 | NP_070039.1 | 11498810 | Archaeoglobus fulgidus |
| AF1983 | NP_070807.1 | 11499565 | Archaeoglobus fulgidus |
| scs | YP_135572.1 | 55377722 | Haloarcula marismortui |
| PAE3250 | NP_560604.1 | 18313937 | Pyrobaculum aerophilum |
| | | | str. IM2 |
| sucC | NP_415256.1 | 16128703 | Escherichia coli |
| sucD | AAC73823.1 | 1786949 | Escherichia coli |
| paaF | AAC24333.2 | 22711873 | Pseudomonas putida |
| matB | AAC83455.1 | 3982573 | Rhizobium leguminosarum |

Several acyl-CoA dehydrogenases are capable of reducing an acyl-CoA to its corresponding aldehyde and can be used for catalyzing the glycolyl-CoA reductase (aldehyde forming) activity. Exemplary genes that encode such enzymes include the Acinetobacter calcoaceticus acr1 encoding a fatty acyl-CoA reductase (Reiser et al., J. Bacteriol. 179:2969-2975 (1997)), the Acinetobacter sp. M-1 fatty acyl-CoA reductase (Ishige et al., Appl. Environ. Microbiol. 68:1192-1195 (2002)), and a CoA- and NADP-dependent succinate semialdehyde dehydrogenase encoded by the sucD gene in Clostridium kluyveri (Sohling et al., J. Bacteriol. 178:871-880 (1996)). SucD of P. gingivalis is another succinate semialdehyde dehydrogenase (Takahashi et al., J. Bacteriol. 182:4704-4710 (2000)). The enzyme acylating acetaldehyde dehydrogenase in Pseudomonas sp, encoded by bphG, is yet another as it has been demonstrated to oxidize and acylate acetaldehyde, propionaldehyde, butyraldehyde, isobutyraldehyde and formaldehyde (Powlowski et al., 175:377-385 (1993)). In addition to reducing acetyl-CoA to ethanol, the enzyme encoded by adhE in Leuconostoc mesenteroides has been shown to oxidize the branched chain compound isobutyraldehyde to isobutyryl-CoA (Koo et al., Biotechnol Lett. 27:505-510 (2005)). Butyraldehyde dehydrogenase catalyzes a similar reaction, conversion of butyryl-CoA to butyraldehyde, in solventogenic organisms such as Clostridium saccharoperbutylacetonicum (Kosaka et al., Biosci. Biotechnol Biochem. 71:58-68 (2007)).

| Protein | GenBank ID | GI Number | Organism |
|---|---|---|---|
| acr1 | YP_047869.1 | 50086359 | Acinetobacter calcoaceticus |
| acr1 | AAC45217 | 1684886 | Acinetobacter baylyi |
| acr1 | BAB85476.1 | 18857901 | Acinetobacter sp. Strain M-1 |
| sucD | P38947.1 | 172046062 | Clostridium kluyveri |
| sucD | NP_904963.1 | 34540484 | Porphyromonas gingivalis |
| bphG | BAA03892.1 | 425213 | Pseudomonas sp |
| adhE | AAV66076.1 | 55818563 | Leuconostoc mesenteroides |
| bld | AAP42563.1 | 31075383 | Clostridium |
| | | | saccharoperbutylacetonicum |

An additional enzyme type that converts an acyl-CoA to its corresponding aldehyde is malonyl-CoA reductase which transforms malonyl-CoA to malonic semialdehyde. Malonyl-CoA reductase is a key enzyme in autotrophic carbon fixation via the 3-hydroxypropionate cycle in thermoacidophilic archael bacteria (Berg et al., Science 318:1782-1786 (2007); Thauer, Science 318:1732-1733 (2007)). The enzyme utilizes NADPH as a cofactor and has been characterized in Metallosphaera and Sulfolobus spp (Alber et al., J. Bacteriol. 188:8551-8559 (2006); Hugler et al., J. Bacteriol. 184:2404-2410 (2002)). The enzyme is encoded by Msed_{—}0709 in Metallosphaera sedula (Alber et al., supra; Berg et al., supra). A gene encoding a malonyl-CoA reductase from Sulfolobus tokodaii was cloned and heterologously expressed in E. coli (Alber et al., supra). This enzyme has also been shown to catalyze the conversion of methylmalonyl-CoA to its corresponding aldehyde (WO/2007/141208). Although the aldehyde dehydrogenase functionality of these enzymes is similar to the bifunctional dehydrogenase from Chloroflexus aurantiacus, there is little sequence similarity. Both malonyl-CoA reductase enzyme candidates have high sequence similarity to aspartate-semialdehyde dehydrogenase, an enzyme catalyzing the reduction and concurrent dephosphorylation of aspartyl-4-phosphate to aspartate semialdehyde. Additional gene candidates can be found by sequence homology to proteins in other organisms including Sulfolobus solfataricus and Sulfolobus acidocaldarius. Yet another acyl-CoA reductase (aldehyde forming) candidate is the ald gene from Clostridium beijerinckii (Toth et al., Appl Environ. Microbiol 65:4973-4980 (1999)). This enzyme has been reported to reduce acetyl-CoA and butyryl-CoA to their corresponding aldehydes. This gene is very similar to eutE that encodes acetaldehyde dehydrogenase of Salmonella typhimurium and E. coli (Toth et al., supra).

| Protein | GenBank ID | GI Number | Organism |
|---|---|---|---|
| MSED_0709 | YP_001190808.1 | 146303492 | Metallosphaera sedula |
| mcr | NP_378167.1 | 15922498 | Sulfolobus tokodaii |
| asd-2 | NP_343563.1 | 15898958 | Sulfolobus solfataricus |
| Saci_2370 | YP_256941.1 | 70608071 | Sulfolobus |
| | | | acidocaldarius |
| Ald | AAT66436 | 9473535 | Clostridium |
| | | | beijerinckii |
| eutE | AAA80209 | 687645 | Salmonella |
| | | | typhimurium |
| eutE | P77445 | 2498347 | Escherichia coli |

Direct conversion of glycolate to glycolaldehyde is catalyzed by an acid reductase enzyme with glycolate reductase activity. Exemplary enzymes include carboxylic acid reductase, alpha-aminoadipate reductase and retinoic acid reductase. Carboxylic acid reductase (CAR), found in Nocardia iowensis, catalyzes the magnesium, ATP and NADPH-dependent reduction of carboxylic acids to their corresponding aldehydes (Venkitasubramanian et al., J Biol. Chem. 282:478-485 (2007)). The natural substrate of this enzyme is vanillic acid and the enzyme exhibits broad acceptance of aromatic and aliphatic substrates (Venkitasubramanian et al., 425-440 (2006)). This enzyme, encoded by car, was cloned and functionally expressed in E. coli (Venkitasubramanian et al., J Biol. Chem. 282:478-485 (2007)). CAR requires post-translational activation by a phosphopantetheine transferase (PPTase) that converts the inactive apo-enzyme to the active holo-enzyme (Hansen et al., Appl. Environ. Microbiol 75:2765-2774 (2009)). Expression of the npt gene, encoding a specific PPTase, product improved activity of the enzyme. An additional enzyme candidate found in Streptomyces griseus is encoded by the griC and griD genes. This enzyme is believed to convert 3-amino-4-hydroxybenzoic acid to 3-amino-4-hydroxybenzaldehyde as deletion of either griC or griD led to accumulation of extracellular 3-acetylamino-4-hydroxybenzoic acid, a shunt product of 3-amino-4-hydroxybenzoic acid metabolism (Suzuki, et al., J. Antibiot. 60(6):380-387 (2007)). Co-expression of griC and griD with SGR_{—}665, an enzyme similar in sequence to the Nocardia iowensis npt, can be beneficial.

| Protein | GenBank ID | GI Number | Organism |
|---|---|---|---|
| car | AAR91681.1 | 40796035 | Nocardia iowensis |
| npt | ABI83656.1 | 114848891 | Nocardia iowensis |
| griC | YP_001825755.1 | 182438036 | Streptomyces griseus |
| griD | YP_001825756.1 | 182438037 | Streptomyces griseus |

An enzyme with similar characteristics, alpha-aminoadipate reductase (AAR, EC 1.2.1.31), participates in lysine biosynthesis pathways in some fungal species. This enzyme naturally reduces alpha-aminoadipate to alpha-aminoadipate semialdehyde. The carboxyl group is first activated through the ATP-dependent formation of an adenylate that is then reduced by NAD(P)H to yield the aldehyde and AMP. Like CAR, this enzyme utilizes magnesium and requires activation by a PPTase. Enzyme candidates for AAR and its corresponding PPTase are found in Saccharomyces cerevisiae (Morris et al., Gene 98:141-145 (1991)), Candida albicans (Guo et al., Mol. Genet. Genomics 269:271-279 (2003)), and Schizosaccharomyces pombe (Ford et al., Curr. Genet. 28:131-137 (1995)). The AAR from S. pombe exhibited significant activity when expressed in E. coli (Guo et al., Yeast 21:1279-1288 (2004)). The AAR from Penicillium chrysogenum accepts S-carboxymethyl-L-cysteine as an alternate substrate, but did not react with adipate, L-glutamate or diaminopimelate (Hijarrubia et al., J Biol. Chem. 278:8250-8256 (2003)). The gene encoding the P. chrysogenum PPTase has not been identified to date and no high-confidence hits were identified by sequence comparison homology searching.

| Protein | GenBank ID | GI Number | Organism |
|---|---|---|---|
| LYS2 | AAA34747.1 | 171867 | Saccharomyces cerevisiae |
| LYS5 | P50113.1 | 1708896 | Saccharomyces cerevisiae |
| LYS2 | AAC02241.1 | 2853226 | Candida albicans |
| LYS5 | AAO26020.1 | 28136195 | Candida albicans |
| Lys1p | P40976.3 | 13124791 | Schizosaccharomyces pombe |
| Lys7p | Q10474.1 | 1723561 | Schizosaccharomyces pombe |
| Lys2 | CAA74300.1 | 3282044 | Penicillium chrysogenum |

Kinase or phosphotransferase enzymes transform carboxylic acids to phosphonic acids with concurrent hydrolysis of one ATP. Such an enzyme with glycolate kinase activity is required to convert glycolate to glycoylylphosphate (FIG. 3, Step 7). This exact transformation has not been demonstrated to date. Exemplary enzyme candidates include butyrate kinase (EC 2.7.2.7), isobutyrate kinase (EC 2.7.2.14), aspartokinase (EC 2.7.2.4), acetate kinase (EC 2.7.2.1) and gamma-glutamyl kinase (EC 2.7.2.11). Butyrate kinase catalyzes the reversible conversion of butyryl-phosphate to butyrate during acidogenesis in Clostridial species (Cary et al., Appl. Environ. Microbiol 56:1576-1583 (1990)). The Clostridium acetobutylicum enzyme is encoded by either of the two buk gene products (Huang et al., J Mol. Microbiol Biotechnol 2:33-38 (2000)). Other butyrate kinase enzymes are found in C. butyricum and C. tetanomorphum (TWAROG et al., J Bacteriol. 86:112-117 (1963)). A related enzyme, isobutyrate kinase from Thermotoga maritima, was expressed in E. coli and crystallized (Diao et al., J Bacteriol. 191:2521-2529 (2009); Diao et al., Acta Crystallogr. D. Biol. Crystallogr. 59:1100-1102 (2003)). Aspartokinase catalyzes the ATP-dependent phosphorylation of aspartate and participates in the synthesis of several amino acids. The aspartokinase III enzyme in E. coli, encoded by lysC, has a broad substrate range and the catalytic residues involved in substrate specificity have been elucidated (Keng et al., Arch. Biochem. Biophys. 335:73-81 (1996)). Two additional kinases in E. coli are also good candidates: acetate kinase and gamma-glutamyl kinase. The E. coli acetate kinase, encoded by ackA (Skarstedt et al., J. Biol. Chem. 251:6775-6783 (1976)), phosphorylates propionate in addition to acetate (Hesslinger et al., Mol. Microbiol 27:477-492 (1998)). The E. coli gamma-glutamyl kinase, encoded by proB (Smith et al., J. Bacteriol. 157:545-551 (1984a)), phosphorylates the gamma carbonic acid group of glutamate.

| Protein | GenBank ID | GI Number | Organism |
|---|---|---|---|
| buk1 | NP_349675 | 15896326 | Clostridium acetobutylicum |
| buk2 | Q97II1 | 20137415 | Clostridium acetobutylicum |
| buk2 | Q9X278.1 | 6685256 | Thermotoga maritima |
| lysC | NP_418448.1 | 16131850 | Escherichia coli |
| ackA | NP_416799.1 | 16130231 | Escherichia coli |
| proB | NP_414777.1 | 16128228 | Escherichia coli |

An enzyme with phosphotransglycolylase activity is required to convert glycolylphosphate to glycolyl-CoA (FIG. 3, Step 8). Exemplary phosphate-transferring acyltransferases include phosphotransacetylase (EC 2.3.1.8) and phosphotransbutyrylase (EC 2.3.1.19). The pta gene from E. coli encodes a phosphotransacetylase that reversibly converts acetyl-CoA into acetyl-phosphate (Suzuki, Biochim. Biophys. Acta 191:559-569 (1969)). This enzyme can also utilize propionyl-CoA as a substrate, forming propionate in the process (Hesslinger et al., Mol. Microbiol 27:477-492 (1998)). Other phosphate acetyltransferases that exhibit activity on propionyl-CoA are found in Bacillus subtilis (Rado et al., Biochim. Biophys. Acta 321:114-125 (1973)), Clostridium kluyveri (Stadtman, 1:596-599 (1955)), and Thermotoga maritima (Bock et al., J Bacteriol. 181:1861-1867 (1999)). Similarly, the ptb gene from C. acetobutylicum encodes phosphotransbutyrylase, an enzyme that reversibly converts butyryl-CoA into butyryl-phosphate (Wiesenborn et al., Appl Environ. Microbiol 55:317-322 (1989); Walter et al., Gene 134:107-111 (1993)). Additional ptb genes are found in butyrate-producing bacterium L2-50 (Louis et al., J. Bacteriol. 186:2099-2106 (2004)) and Bacillus megaterium (Vazquez et al., Curr. Microbiol 42:345-349 (2001)).

| Protein | GenBank ID | GI Number | Organism |
|---|---|---|---|
| pta | NP_416800.1 | 71152910 | Escherichia coli |
| pta | P39646 | 730415 | Bacillus subtilis |
| pta | A5N801 | 146346896 | Clostridium kluyveri |
| pta | Q9X0L4 | 6685776 | Thermotoga maritima |
| ptb | NP_349676 | 34540484 | Clostridium acetobutylicum |
| ptb | AAR19757.1 | 38425288 | butyrate-producing bacterium |
| | | | L2-50 |
| ptb | CAC07932.1 | 10046659 | Bacillus megaterium |

The conversion of glycolylphosphate to glycolaldehyde is catalyzed by glycolylphosphate reductase (FIG. 3, Step 9). Although an enzyme catalyzing this conversion has not been identified to date, similar transformations catalyzed by glyceraldehyde-3-phosphate dehydrogenase (EC 1.2.1.12), aspartate-semialdehyde dehydrogenase (EC 1.2.1.11) acetylglutamylphosphate reductase (EC 1.2.1.38) and glutamate-5-semialdehyde dehydrogenase (EC 1.2.1.) are well documented. Aspartate semialdehyde dehydrogenase (ASD, EC 1.2.1.11) catalyzes the NADPH-dependent reduction of 4-aspartyl phosphate to aspartate-4-semialdehyde. ASD participates in amino acid biosynthesis and recently has been studied as an antimicrobial target (Hadfield et al., Biochemistry 40:14475-14483 (2001)). The E. coli ASD structure has been solved (Hadfield et al., J Mol. Biol. 289:991-1002 (1999)) and the enzyme has been shown to accept the alternate substrate beta-3-methylaspartyl phosphate (Shames et al., J Biol. Chem. 259:15331-15339 (1984)). The Haemophilus influenzae enzyme has been the subject of enzyme engineering studies to alter substrate binding affinities at the active site (Blanco et al., Acta Crystallogr. D. Biol. Crystallogr. 60:1388-1395 (2004)). Other ASD candidates are found in Mycobacterium tuberculosis (Shafiani et al., J Appl Microbiol 98:832-838 (2005)), Methanococcus jannaschii (Faehnle et al., J Mol. Biol. 353:1055-1068 (2005)), and the infectious microorganisms Vibrio cholera and Helicobacter pylori (Moore et al., Protein Expr. Purif. 25:189-194 (2002)). A related enzyme candidate is acetylglutamylphosphate reductase (EC 1.2.1.38), an enzyme that naturally reduces acetylglutamylphosphate to acetylglutamate-5-semialdehyde, found in S. cerevisiae (Pauwels et al., Eur. J Biochem. 270:1014-1024 (2003)), B. subtilis (O'Reilly et al., Microbiology 140 (Pt 5):1023-1025 (1994)), E. coli (Parsot et al., Gene. 68:275-283 (1988)), and other organisms. Additional phosphate reductase enzymes of E. coli include glyceraldehyde 3-phosphate dehydrogenase encoded by gapA (Branlant et al., Eur. J. Biochem. 150:61-66 (1985)) and glutamate-5-semialdehyde dehydrogenase encoded by proA (Smith et al., J. Bacteriol. 157:545-551 (1984b)). Genes encoding glutamate-5-semialdehyde dehydrogenase enzymes from Salmonella typhimurium (Mahan et al., J Bacteriol. 156:1249-1262 (1983)) and Campylobacter jejuni (Louie et al., Mol. Gen. Genet. 240:29-35 (1993)) were cloned and expressed in E. coli.

| Protein | GenBank ID | GI Number | Organism |
|---|---|---|---|
| asd | NP_417891.1 | 16131307 | Escherichia coli |
| asd | YP_248335.1 | 68249223 | Haemophilus influenzae |
| asd | AAB49996 | 1899206 | Mycobacterium tuberculosis |
| VC2036 | NP_231670 | 15642038 | Vibrio cholera |
| asd | YP_002301787.1 | 210135348 | Helicobacter pylori |
| ARG5,6 | NP_010992.1 | 6320913 | Saccharomyces cerevisiae |
| argC | NP_389001.1 | 16078184 | Bacillus subtilis |
| argC | NP_418393.1 | 16131796 | Escherichia coli |
| gapA | P0A9B2.2 | 71159358 | Escherichia coli |
| proA | NP_414778.1 | 16128229 | Escherichia coli |
| proA | NP_459319.1 | 16763704 | Salmonella typhimurium |
| proA | P53000.2 | 9087222 | Campylobacter jejuni |

The direct formation of ethylene glycol from glycolyl-CoA is catalyzed by a bifunctional enzyme with glycolyl-CoA reductase (alcohol forming) activity (FIG. 3, Step 10). Exemplary bifunctional oxidoreductases that convert acyl-CoA molecules to their corresponding alcohols include enzymes that transform substrates such as acetyl-CoA to ethanol (e.g., adhE from E. coli (Kessler et al., FEBS. Lett. 281:59-63 (1991))), butyryl-CoA to butanol (e.g. adhE2 from C. acetobutylicum (Fontaine et al., J. Bacteriol. 184:821-830 (2002))) and malonyl-CoA to 3-hydroxypropanoate (e.g. mcr from Chloroflexus aurantiacus (Hugler et al., J. Bacteriol. 184:2404-2410 (2002))). In addition to reducing acetyl-CoA to ethanol, the enzyme encoded by adhE in Leuconostoc mesenteroides has been shown to oxidize the branched chain compound isobutyraldehyde to isobutyryl-CoA (Kazahaya et al., J. Gen. Appl. Microbiol. 18:43-55 (1972); Koo et al., supra). The NADPH-dependent alcohol-forming malonyl-CoA reductase of Chloroflexus aurantiacus participates in the 3-hydroxypropionate cycle (Hugler et al., 184:2404-2410 (2002); Strauss et al., Eur. J. Biochem. 215:633-643 (1993)). This enzyme, with a mass of 300 kDa, is highly substrate-specific and shows little sequence similarity to other known oxidoreductases (Hugler et al., supra). No enzymes in other organisms have been shown to catalyze this specific reaction; however there is bioinformatic evidence that other organisms may have similar pathways (Klatt et al., supra). Enzyme candidates in other organisms including Roseiflexus castenholzii, Erythrobacter sp. NAP1 and marine gamma proteobacterium HTCC2080 can be inferred by sequence similarity.

| Protein | GenBank ID | GI Number | Organism |
|---|---|---|---|
| adhE | NP_415757.1 | 16129202 | Escherichia coli |
| adhE2 | AAK09379.1 | 12958626 | Clostridium acetobutylicum |
| adhE | AAV66076.1 | 55818563 | Leuconostoc mesenteroides |
| mcr | AAS20429.1 | 42561982 | Chloroflexus aurantiacus |
| Rcas_2929 | YP_001433009.1 | 156742880 | Roseiflexus castenholzii |
| NAP1_02720 | ZP_01039179.1 | 85708113 | Erythrobacter sp. NAP1 |
| MGP2080_00535 | ZP_01626393.1 | 119504313 | marine gamma proteobacterium HTCC2080 |

Longer chain acyl-CoA molecules can be reduced to their corresponding alcohols by enzymes such as the jojoba (Simmondsia chinensis) FAR which encodes an alcohol-forming fatty acyl-CoA reductase. Its overexpression in E. coli resulted in FAR activity and the accumulation of fatty alcohol (Metz et al., Plant Physiol. 122:635-644 (2000)).

| Protein | GenBank ID | GI Number | Organism |
|---|---|---|---|
| FAR | AAD38039.1 | 5020215 | Simmondsia chinensis |

### Example V

### Pathways for Converting Glycerate to Ethylene Glycol

Pathways for converting glycerate to ethylene glycol are shown in FIG. 1. Glycerate is a common metabolic intermediate in diverse metabolic biosynthetic and degradation pathways including the non-phosphorylative Entner-Doudoroff pathway, the serine pathway of formaldehyde assimilation and gloxylate degradation. Glycerate can also be formed by oxidation of glyceraldehyde by glyceraldehyde dehydrogenase or glyceraldehyde oxidase (Step 14 of FIG. 1) or dephosphorylation of 3-phosphoglycerate or 2-phosphoglycerate by either a phosphatase (Steps 10 and 12 of FIG. 1) or a kinase operating in the reverse direction (Steps 13 and 11 of FIG. 1). The glycerate is then converted to ethylene glycol by one of several pathways. In one pathway, glycerate is directly converted to ethylene glycol by a decarboxylase (Step 9 of FIG. 1). Candidate enzymes for this decarboxylase were presented in Example I. In an alternate route, glycerate is oxidized to hydroxypyruvate by hydroxypyruvate reductase (Step 8 of FIG. 1). The hydroxypyruvate intermediate is then decarboxylated and reduced to ethylene glycol by enzymes described in Example I (FIG. 1, Steps 3, 4). In yet another route, glycerate is converted to hydroxypyruvate in two steps: oxidation to tartronate semialdehyde by glycerate dehydrogenase, followed by isomerization to hydroxypyruvate by hydroxypyruvate isomerase (Steps 5 and 2 of FIG. 2). Enzyme candidates for hydroxypyruvate isomerase were described in Example III. Enzyme candidates for 2-phosphoglycerate phosphatase, glycerate-2-kinase, glyceraldehyde dehydrogense, glyceraldehyde oxidase and glycerate dehydrogenase are provided below.

2-Phosphoglycerate phosphatase (EC 3.1.3.20) catalyzes the hydrolysis of 2PG to glycerate, releasing pyrophosphate (FIG. 1, Step 12). This enzyme was purified from cell extracts of Veillonella alcalescens (Pestka et al., Can. J Microbiol 27:808-814 (1981)), where it is thought to participate in a serine biosynthetic pathway. A similar enzyme was also characterized in beef liver (Fallon et al., Biochim Biophys Acta 105:43-53 (1965)). However, genes have not been associated with either enzyme to date. Additional 2PG phosphatase enzyme candidates are alkaline phosphatase (EC 3.1.3.1) and acid phosphatase (EC 3.1.3.2). Both enzymes hydrolyze a broad range of phosphorylated substrates to their corresponding alcohols. Alkaline phosphatase enzymes are typically secreted into the periplasm in bacteria, where they play a role in phosphate transport and metabolism. The E. coli phoA gene encodes a periplasmic zinc-dependent alkaline phosphatase active under conditions of phosphate starvation (Coleman Annu. Rev. Biophys. Biomol. Struct. 21:441-83 (1992)). Similar enzymes have been characterized in Campylobacter jejuni (van Mourik et al., Microbiol. 154:584-92 (2008)), Saccharomyces cerevisiae (Oshima et al., Gene 179:171-7 (1996)) and Staphylococcus aureus (Shah and Blobel, J. Bacteriol. 94:780-1 (1967)). Enzyme engineering and/or removal of targeting sequences may be required for alkaline phosphatase enzymes to function in the cytoplasm. Acid phosphatase enzymes from Brassica nigra, Lupinus luteus and Phaseolus vulgaris have been shown to catalyze the hydrolysis of 2PG to glycerate (Yoneyama et al., J Biol Chem 279:37477-37484 (2004); Olczak et al., Biochim Biophys Acta 1341:14-25 (1997); Duff et al., Arch. Biochem. Biophys 286:226-232 (1991)). Only the P. vulgaris enzyme has been associated with a gene to date.

| Protein | GenBank ID | GI Number | Organism |
|---|---|---|---|
| phoA | NP_414917.2 | 49176017 | Escherichia coli |
| phoX | ZP_01072054.1 | 86153851 | Campylobacter jejuni |
| PHO8 | AAA34871.1 | 172164 | Saccharomyces |
| | | | cerevisiae |
| SaurJH1_2706 | YP_001317815.1 | 150395140 | Staphylococcus aureus |
| KeACP | BAD05167.1 | 40217508 | Phaseolus vulgaris |

The ATP-dependent interconversion of 2-phosphoglycerate and glycerate (FIG. 1, Step 13) is catalyzed by glycerate-2-kinase (EC 2.7.1.165). This enzyme naturally operates in the phosphorylating ATP-consuming direction and has not been shown to function in the reverse ATP-generating direction. Glycerate-2-kinase enzymes have been studied in animals, methylotrophic bacteria and organisms which utilize a branched Entner-Doudoroff pathway. Exemplary gene candidates include glxK of E. coli (Bartsch et al., FEBS Lett. 582:3025-3028 (2008)), ST2037 of Sulfolobus tolodaii, garK of Thermoproteus tenax and Pto1442 from Picrophilus torridus (Liu et al., Biotechnol Lett. 31:1937-1941 (2009); Kehrer et al., BMC Genomics 8:301 (2007); Reher et al., FEMS Microbiol Lett. 259:113-119 (2006)). The thermostable enzymes of S. tolodaii and T. tenax have been cloned and characterized in E. coli. Several enzymes in this class are inhibited by ADP, so removal or attenuation of this inhibition may be necessary for the enzyme to operate in the desired direction.

| Protein | GenBank ID | GI Number | Organism |
|---|---|---|---|
| glxK | AAC73616.1 | 1786724 | Escherichia coli |
| ST2037 | NP_378024.1 | 15922355 | Sulfolobus tolodaii |
| garK | AJ621354 | 41033736 | Thermoproteus tenax |
| Pto1442 | YP_024220.1 | 48478514 | Picrophilus torridus |

Glyceraldehyde dehydrogenase catalyzes the oxidation of glyceraldehyde to glycerate. This reaction can be catalyzed by many NAD(P)+-dependent oxidoreductases in the EC class 1.2.1. Exemplary enzymes that catalyze this conversion include the glutarate semialdehyde dehydrogenase (EC 1.2.1.20) of Pseudomonas putida, lactate dehydrogenase (EC 1.2.1.22) of Methanocaldococcus jannaschii, the betaine-aldehyde dehydrogenase (EC 1.2.1.8) of E. coli (Gruez et al., J Mol Biol 343:29-41 (2004)) and the succinate semialdehyde dehydrogenase (EC 1.2.1.24) of Azospirillum brasilense (Watanabe et al., J Biol Chem 281:28876-28888 (2006); Grochowski et al., J Bacteriol. 188:2836-2844 (2006); Chang et al., J Biol Chem 252:7979-7986 (1977)). The NAD+- and NADP+-dependent aldehyde dehydrogenase enzymes (EC 1.2.1.3 and EC 1.2.1.4 and EC 1.2.1.5) are also suitable candidates. Some gene products with activity on glyceraldehyde include the NADP+ dependent enzyme from Acetobacter aceti and ALDH from Saccharomyces cerevisiae (Vandecasteele et al., Methods Enzymol. 89 Pt D:484-490 (1982); Tamaki et al., J Biochem. 82:73-79 (1977)). The NAD+-dependent aldehyde dehydrogenases (EC 1.2.1.3) found in human liver, ALDH-1 and ALDH-2, have broad substrate ranges for a variety of aliphatic, aromatic and polycyclic aldehydes (Klyosov, Biochemistry 35:4457-4467 (1996)). Active ALDH-2 has been efficiently expressed in E. coli using the GroEL proteins as chaperonins (Lee et al., Biochem. Biophys. Res. Commun. 298:216-224 (2002)). The rat mitochondrial aldehyde dehydrogenase also has a broad substrate range (Siew et al., Arch. Biochem. Biophys. 176:638-649 (1976)). The E. coli gene astD also encodes an NAD+-dependent aldehyde dehydrogenase (Kuznetsova et al., FEMS Microbiol Rev 29:263-279 (2005)).

| Protein | GenBank ID | GI Number | Organism |
|---|---|---|---|
| MJ1411 | NP_248414.1 | 15669601 | Methanocaldococcus |
| | | | jannaschii |
| araE | BAE94276.1 | 95102056 | Azospirillum brasilense |
| ydcW | NP_415961.1 | 16129403 | Escherichia coli |
| ALDH | AAA34419.1 | 171048 | Saccharomyces cerevisiae |
| ALDH-2 | P05091.2 | 118504 | Homo sapiens |
| ALDH-2 | NP_115792.1 | 14192933 | Rattus norvegicus |
| astD | P76217.1 | 3913108 | Escherichia coli |
| PF0346 | NP_578075.1 | 18976718 | Pyrococcus furiosus |

Aldehyde oxidase enzymes (EC 1.2.3.1) can also catalyze the conversion of glyceraldehyde, water and oxygen to glycerate and hydrogen peroxide. Aldehyde oxidase enzymes in organisms such as Streptomyces moderatus, Pseudomonas sp. and Methylobacillus sp. catalyze the oxidation of a wide range of aldehydes including formaldehyde, aromatic, and aliphatic aldehydes including glyceraldehyde (Koshiba et al., Plant Physiol 110:781-789 (1996)). Although the genes associated with these enzymes are not known to date, the zmAO-1 and zmAO-2 genes of Zea mays encode flavin- and molybdenum-containing aldehyde oxidase isozymes (Sekimoto et al., J Biol. Chem. 272:15280-15285 (1997)). Additional glyceraldehyde oxidase candidates can be inferred by sequence homology to the Z. mays genes and are shown below.

| | GenBank | | |
|---|---|---|---|
| Gene | Accession No. | GI No. | Organism |
| zmAO-1 | NP_001105308.1 | 162458742 | Zea mays |
| zmAO-2 | BAA23227.1 | 2589164 | Zea mays |
| Aox1 | O54754.2 | 20978408 | Mus musculus |
| ALDO1_ORYSJ | Q7XH05.1 | 75296231 | Oryza sativa |
| AAO3 | BAA82672.1 | 5672672 | Arabidopsis |
| | | | thaliana |
| XDH | DAA24801.1 | 296482686 | Bos taurus |

Oxidation of glycerate to tartronate semialdehyde is catalyzed by glycerate dehydrogenase (EC 1.1.1.60). Two isozymes of this enzyme are encoded by the genes garR and glxR of E. coli (Cusa et al., J. Bacteriol. 181:7479-7484 (1999); Monterrubio et al., J. Bacteriol. 182:2672-2674 (2000); Njau et al., J Biol Chem 275:38780-38786 (2000)). The glycerate dehydrogenase encoded by garR of Salmonella typhimurium subsp. enterica serovar Typhimurium was recently crystallized (Osipiuk et al., J. Struct. Funct. Genomics 10:249-253 (2009)).

| | GenBank | | |
|---|---|---|---|
| Gene | Accession No. | GI No. | Organism |
| garR | AAC76159.3 | 145693186 | Escherichia coli |
| glxR | AAC73611.1 | 1786719 | Escherichia coli |
| garR | NP_462161.1 | 16766546 | Salmonella typhimurium |

Additional candidate alcohol dehydrogenases for converting glycerate to tartronate semialdehyde include medium-chain alcohol dehydrogenase, 4-hydroxybutyrate dehydrogenase and 3-hydroxyisobutyrate dehydrogenase. Exemplary genes encoding medium-chain alcohol dehydrogenase enzymes that catalyze the conversion of an alcohol to an aldehyde include alrA encoding a medium-chain alcohol dehydrogenase for C₂-C₁₄ (Tani et al., Appl. Environ. Microbiol. 66:5231-5235 (2000)), ADH2 from Saccharomyces cerevisiae (Atsumi et al., Nature 451:86-89 (2008)), yqhD from E. coli which has preference for molecules longer than C(3) (Sulzenbacher et al., 342:489-502 (2004)), and bdh I and bdh II from C. acetobutylicum which converts butyryaldehyde into butanol (Walter et al., 174:7149-7158 (1992)). The gene product of yqhD catalyzes the reduction of acetaldehyde, malondialdehyde, propionaldehyde, butyraldehyde, and acrolein using NADPH as the cofactor (Perez et al., J Biol. Chem. 283:7346-7353 (2008a); Perez et al., J Biol. Chem. 283:7346-7353 (2008b)). The adhA gene product from Zymomonas mobilis has been demonstrated to have activity on a number of aldehydes including formaldehyde, acetaldehyde, propionaldehyde, butyraldehyde, and acrolein (Kinoshita et al., Appl Microbiol Biotechnol 22:249-254 (1985)). Additional alcohol dehydrogenase candidates are encoded by bdh in C. saccharoperbutylacetonicum and Cbei_{—}1722, Cbei_{—}2181 and Cbei_{—}2421 in C. beijerinckii.

| | GenBank | | |
|---|---|---|---|
| Gene | Accession No. | GI No. | Organism |
| alrA | BAB12273.1 | 9967138 | Acinetobacter sp. strain M-1 |
| ADH2 | NP_014032.1 | 6323961 | Saccharomyces cerevisiae |
| yqhD | NP_417484.1 | 16130909 | Escherichia coli |
| bdh I | NP_349892.1 | 15896543 | Clostridium acetobutylicum |
| bdh II | NP_349891.1 | 15896542 | Clostridium acetobutylicum |
| adhA | YP_162971.1 | 56552132 | Zymomonas mobilis |
| bdh | BAF45463.1 | 124221917 | Clostridium saccharoperbutylacetonicum |
| Cbei_1722 | YP_001308850 | 150016596 | Clostridium beijerinckii |
| Cbei_2181 | YP_001309304 | 150017050 | Clostridium beijerinckii |
| Cbei_2421 | YP_001309535 | 150017281 | Clostridium beijerinckii |

Enzymes exhibiting 4-hydroxybutyrate dehydrogenase activity (EC 1.1.1.61) have been characterized in Ralstonia eutropha (Bravo et al., J. Forensic Sci. 49:379-387 (2004)), Clostridium kluyveri (Wolff et al., Protein Expr. Purif. 6:206-212 (1995)) and Arabidopsis thaliana (Breitkreuz et al., J. Biol. Chem. 278:41552-41556 (2003)). The A. thaliana enzyme was cloned and characterized in yeast (Breitkreuz et al., J. Biol. Chem. 278:41552-41556 (2003)). Yet another gene is the alcohol dehydrogenase adhI from Geobacillus thermoglucosidasius (Jeon et al., J Biotechnol 135:127-133 (2008)).

| | GenBank | | |
|---|---|---|---|
| Gene | Accession No. | GI No. | Organism |
| 4hbd | YP_726053.1 | 113867564 | Ralstonia eutropha H16 |
| 4hbd | L21902.1 | 146348486 | Clostridium kluyveri DSM 555 |
| 4hbd | Q94B07 | 75249805 | Arabidopsis thaliana |
| adhI | AAR91477.1 | 40795502 | Geobacillus thermoglucosidasius |

3-Hydroxyisobutyrate dehydrogenase (EC 1.1.1.31) catalyzes the reversible oxidation of 3-hydroxyisobutyrate to methylmalonate semialdehyde. This enzyme participates in valine, leucine and isoleucine degradation and has been identified in bacteria, eukaryotes, and mammals. The enzyme encoded by P84067 from Thermus thermophilus HB8 has been structurally characterized (Lokanath et al., 352:905-17 (2005)). Additional genes encoding this enzyme include 3hidh in Homo sapiens (Hawes et al., 324:218-228 (2000)) and Oryctolagus cuniculus (Hawes et al., Methods Enzymol. 324:218-228 (2000); Chowdhury et al., Biosci. Biotechnol Biochem. 60:2043-2047 (1996)), mmsB in Pseudomonas aeruginosa and Pseudomonas putida, and dhat in Pseudomonas putida (Aberhart et al., J Chem. Soc. [Perkin 1] 6:1404-1406 (1979); Chowdhury et al., Biosci. Biotechnol Biochem. 60:2043-2047 (1996); Chowdhury et al., Biosci. Biotechnol Biochem. 67:438-441 (2003)).

| | GenBank | | |
|---|---|---|---|
| Gene | Accession No. | GI No. | Organism |
| P84067 | P84067 | 75345323 | Thermus thermophilus |
| 3hidh | P31937.2 | 12643395 | Homo sapiens |
| 3hidh | P32185.1 | 416872 | Oryctolagus cuniculus |
| mmsB | NP_746775.1 | 26991350 | Pseudomonas putida |
| mmsB | P28811.1 | 127211 | Pseudomonas aeruginosa |
| dhat | Q59477.1 | 2842618 | Pseudomonas putida |

Throughout this application various publications have been referenced. The disclosures of these publications in their entireties, including GenBank and GI number publications, are hereby incorporated by reference in this application in order to more fully describe the state of the art to which this invention pertains. Although the invention has been described with reference to the examples provided above, it should be understood that various modifications can be made without departing from the spirit of the invention.

1. A non-naturally occurring microbial organism, comprising a microbial organism having an ethylene glycol pathway comprising at least one exogenous nucleic acid encoding an ethylene glycol pathway enzyme expressed in a sufficient amount to produce ethylene glycol, said ethylene glycol pathway comprising a serine decarboxylase, a serine aminotransferase, a serine oxidoreductase (deaminating), a hydroxypyruvate decarboxylase, a glycolaldehyde reductase, an ethanolamine aminotransferase, an ethanolamine oxidoreductase (deaminating), a hydroxypyruvate reductase or a glycerate decarboxylase.

2. The non-naturally occurring microbial organism of claim 1, wherein said microbial organism comprises two, three, four, five, six, seven, eight or nine exogenous nucleic acids each encoding an ethylene glycol pathway enzyme.

3. The non-naturally occurring microbial organism of claim 1, wherein said ethylene glycol pathway comprises a serine aminotransferase or a serine oxidoreductase (deaminating); a hydroxypyruvate decarboxylase, and a glycolaldehyde reductase.

4. The non-naturally occurring microbial organism of claim 1, wherein said ethylene glycol pathway comprises a serine aminotransferase or a serine oxidoreductase (deaminating); a hydroxypyruvate reductase, and a glycerate decarboxylase.

5. The non-naturally occurring microbial organism of claim 1, wherein said ethylene glycol pathway comprises a serine decarboxylase; an ethanolamine aminotransferase or an ethanolamine oxidoreductase (deaminating), and a glycolaldehyde reductase.

6. The non-naturally occurring microbial organism of claim 1, wherein said at least one exogenous nucleic acid is a heterologous nucleic acid.

7. The non-naturally occurring microbial organism of claim 1, wherein said non-naturally occurring microbial organism is in a substantially anaerobic culture medium.

8. A non-naturally occurring microbial organism, comprising a microbial organism having an ethylene glycol pathway comprising at least one exogenous nucleic acid encoding an ethylene glycol pathway enzyme expressed in a sufficient amount to produce ethylene glycol, said ethylene glycol pathway comprising a hydroxypyruvate decarboxylase, a glycolaldehyde reductase, a hydroxypyruvate reductase, a glycerate decarboxylase, a 3-phosphoglycerate phosphatase, a glycerate kinase, a 2-phosphoglycerate phosphatase, a glycerate-2-kinase or a glyceraldyhyde dehydrogenase.

9. The non-naturally occurring microbial organism of claim 8, wherein said microbial organism comprises two, three, four, five, six, seven, eight or nine exogenous nucleic acids each encoding an ethylene glycol pathway enzyme.

10. The non-naturally occurring microbial organism of claim 8, wherein said ethylene glycol pathway comprises a hydroxypyruvate reductase; a hydroxypyruvate decarboxylase, and a glycolaldehyde reductase.

11. The non-naturally occurring microbial organism of claim 8, wherein said ethylene glycol pathway comprises a glycerate decarboxylase.

12. The non-naturally occurring microbial organism of claim 10, wherein said ethylene glycol pathway further comprises a 3-phosphoglycerate phosphatase or a glycerate kinase.

13. The non-naturally occurring microbial organism of claim 10, wherein said ethylene glycol pathway further comprises a 2-phosphoglycerate phosphatase or a glycerate-2-kinase.

14. The non-naturally occurring microbial organism of claim 10, wherein said ethylene glycol pathway further comprises a glyceraldehyde dehydrogenase.

15. The non-naturally occurring microbial organism of claim 8, wherein said at least one exogenous nucleic acid is a heterologous nucleic acid.

16. The non-naturally occurring microbial organism of claim 8, wherein said non-naturally occurring microbial organism is in a substantially anaerobic culture medium.

17-33. (canceled)

34. A method for producing ethylene glycol, comprising culturing the non-naturally occurring microbial organism of claim 1 under conditions and for a sufficient period of time to produce ethylene glycol.

35. (canceled)

36. A method for producing ethylene glycol, comprising culturing the non-naturally occurring microbial organism of claim 8 under conditions and for a sufficient period of time to produce ethylene glycol.

37. The non-naturally occurring microbial organism of claim 11, wherein said ethylene glycol pathway further comprises a 3-phosphoglycerate phosphatase or a glycerate kinase.

38. The non-naturally occurring microbial organism of claim 11, wherein said ethylene glycol pathway further comprises a 2-phosphoglycerate phosphatase or a glycerate-2-kinase.

39. The non-naturally occurring microbial organism of claim 11, wherein said ethylene glycol pathway further comprises a glyceraldehyde dehydrogenase.
